(19) United States
(12) Patent Application Publication
(10) Pub. No.: US 2009/0142805 A1
(43) Pub. Date: Jun. 4, 2009

(54) High expression cell line that eliminates gene amplification

(75) Inventors: Anthony DiLeo, Westford, MA (US); William Kopaciewicz, West Newbury, MA (US)

Correspondence Address: MILLIPORE CORPORATION, 290 CONCORD ROAD, BILLERICA, MA, 01821, US

(73) Assignee: Millipore Corporation, Billerica, MA (US)

(21) Appl. No.: 12/006,960

(22) Filed: Jan. 8, 2008

Related U.S. Application Data
(60) Provisional application No. 60/879,162, filed on Jan. 8, 2007.

(51) Int. Cl. C12P 21/08; C12P 21/00; C12M 1/00

(52) U.S. Cl. 435/69.6; 435/69.1; 435/283.1; 435/285.2

(57) ABSTRACT

The present invention relates to methods, cell lines and kits for producing high titers of recombinant proteins without the need for gene amplification.

### RELATED APPLICATIONS

This application claims the benefit of priority of U.S. Provisional Patent Application No. 60/872,162, filed on Jan. 8, 2007, the entire contents of which are incorporated by reference herein.

This application is related to U.S. Provisional Patent Application No. 60/897,221, also filed on Jan. 8, 2007, and U.S. Application having attorney docket no. MCA-844 US, filed on Jan. 8, 2008, and entitled “Cell Culture Methods For Producing Recombinant Proteins In The Presence Of Reduced Levels Of One Or More Contaminants,” the entire contents of each of which are incorporated by reference herein.

The entire contents of each of these patent applications are hereby expressly incorporated herein by reference including without limitation the specification, claims, and abstract, as well as any figures, tables, or drawings thereof.

### DESCRIPTION OF THE INVENTION

1. Field of the Invention

The invention relates generally to the field of recombinant protein expression technology. More specifically, the present invention provides methods, cell lines, and kits for producing high titers of recombinant proteins in cell culture, without the need for gene amplification.

2. Background of the Invention

Production of recombinant proteins suitable for use as therapeutics, diagnostic and/or research reagents is known within the Biotechnology field. Typically, the process ranges from identifying a stable clone, which produces a desirable product, to scaling up the manufacturing and product purification, and typically is arduous and lengthy, demanding a significant commitment of time, labor and resources.

Stable gene expression is achieved through the insertion of recombinant gene(s) into the host genome. However, the identification and characterization of recombinant cell lines is a costly and time-consuming process. One significant limiting step in this process is the identification and selection of stably transfected clones that express the target protein at high production rates, approaching 40-50 pg/cell/day. Usually the process involves several rounds of gene amplification process using a selection marker in order to identify clones that express optimal amounts of the target protein, thereby making the whole process time consuming and laborious.

Therefore, a need exists for a simplified, more efficient method of producing target products in cell culture.

### SUMMARY OF THE INVENTION

In certain embodiments the invention relates to improved methods, cell lines and kits for producing a high titer of recombinant proteins in cell culture, without the need for gene amplification.

In some embodiments, a method of producing a high titer of a recombinant protein without the need for gene amplification is provided. The method comprises introducing into one or more cells, a nucleic acid molecule comprising one or more DNA elements capable of opening chromatin and/or maintaining chromatin in an open state operably linked to a nucleotide sequence encoding the recombinant protein, where the nucleic acid molecule is introduced into the one or more cells using high efficiency transfection.

High efficiency transfection comprises introduction of a nucleic acid molecule into at least 50% or more, or at least 60% or more, or at least 70% or more, or at least 75% or more, or at least 80% or more, or at least 85% or more, or at least 90% or more, or at least 95% or more, or at least 96% or more, or at least 97% or more, or at least 98% or more, or at least 99% or more or 100% of the cells being transfected using the methods of the invention.

In one aspect, the high efficiency transfection comprises controlled electroporation which comprises the steps of: 1) placing the one or more cells in an electroporation device comprising a barrier having one or more openings suitable for receiving the cell; 2) securing the one or more cells in the one or more openings; 3) contacting the one or more cells with the nucleic acid molecule; 4) contacting the one or more cells with an electric current such that the current passes through the one or more cells; 5) monitoring the ratio between the current and voltage in the electroporation device; and 6) adjusting the magnitude of the local field strength to a field strength suitable to achieve electroporation of the one or more cells.

In some embodiments, the one or more cells are contacted with the nucleic acid molecule before they are contacted with the electric current. In other embodiments, the one or more cells are contacted with the electric current before they are contacted with the nucleic acid molecule. In still other embodiments, the one or more cells are contacted with the nucleic acid molecule concurrently with the electric current.

In some embodiments, the barrier comprises a dielectric material. In some embodiments, the diameter of the one or more openings is smaller than the diameter of the one or more cells. In other embodiments, the diameter of the one or more openings is substantially the same as the diameter of the one or more cells. In some embodiments, at least 80%, or at least 90, or at least 95%, or at least 96%, or at least 97%, or at least 98%, or at least 99%, or 100% of the one or more openings are plugged by the one or more cells being electroporated.

In some embodiments, the one or more cells are secured to the one or more openings by the application of pressure. In other embodiments, the one or more cells are secured to the opening by the application of vacuum.

In some embodiments, the electroporation device comprises two chambers, each suitable for receiving a buffer. Each chamber may include the same buffer or a different buffer.

In another aspect, the high efficiency transfection comprises controlled electroporation comprising the steps of: 1) placing the one or more cells in an electroporation device comprising at least one elongate capillary having a lumen comprising a first end and a second end, wherein both the first end and the second end open into reservoirs and wherein the one or more cells can flow through the lumen of the at least one capillary and into the reservoirs; 2) contacting the one or more cells with a nucleic acid molecule comprising one or more DNA elements capable of opening chromatin and/or maintaining chromatin in an open state operably linked to a nucleotide sequence encoding the recombinant protein; 3) contacting the one or more cells with an electric current such that the current passes through the one or more cells; 4) monitoring the ratio between the current and voltage in the electroporation device; and 5) adjusting the magnitude of the local field strength to a field strength suitable to achieve electroporation of the one or more cells.

In some embodiments, the one or more cells are contacted with the nucleic acid molecule before they are contacted with the electric current. In other embodiments, the one or more cells are contacted with the electric current before they are contacted with the nucleic acid molecule. In still other embodiments, the one or more cells are contacted with the nucleic acid molecule concurrently with the electric current.

In some embodiments, the diameter of the one or more cells is at least 80%, or at least 90%, or at least 95%, or at least 96%, or at least 97%, or at least 98%, or at least 99%, of the diameter of the lumen of the one or more capillaries. In other embodiments, the diameter of the one or more cells is greater than the diameter of the lumen of the one or more capillaries. In still other embodiments, a plurality of cells occupy at least 80% or more of the total area inside the lumen of the one or more capillaries.

Conversely, in some embodiments, the diameter of the lumen of the at least one or more capillaries is at least 20% greater than the diameter of the one or more cells. In other embodiments, the diameter of the lumen of the one or more capillaries is at least 20% greater than the diameter around the perimeter of a plurality of cells inside the lumen of the one or more capillaries.

In some embodiments according to the various aspects of the invention, the electric field strength is about 150-500 V/cm. In other embodiments, the electric field strength is about 200-400 V/cm. In still other embodiments, the electric field strength is about 250-350 V/cm. In a particular embodiment, the electric field strength is about 400 V/cm.

In some embodiments, nanoparticles or magnetic nanoparticles are used for high efficiency transfection.

In some embodiments, the nucleic acid molecule is a vector, e.g., a plasmid or a viral vector.

In some embodiments, the one or more cells transfected using the methods of the invention are mammalian cells. Exemplary mammalian cells include, but are not limited to, a BHK21 cell, a CHO cell, a CHO-K1 cell, a CHO-DUXX cell, an NSO cell or an Sp2/0 cell. In a particular embodiment, the mammalian cell is a Chinese Hamster Ovary Cell (CHO cell).

In some embodiments, the recombinant protein is a therapeutic protein. In other embodiments, the recombinant protein is an antibody (e.g., a monoclonal antibody) or an antigen-binding fragment thereof.

In some embodiments, the one or more DNA elements capable of opening chromatin and/or maintaining the chromatin in an open state are chosen from: (a) one or more an extended methylation-free CpG islands; (b) one or more matrix attachment regions; (c) one or more stabilizing and antirepressor regions; and (d) any combinations of (a)-(c).

In some embodiments, one or more extended methylation-free CpG islands are derived from the promoter region of one or more ubiquitously expressed genes. Exemplary ubiquitously expressed genes include, but are not limited to, human hnRNPA2, mouse hnRNPA2, human TBP, mouse TBP, human rpS3 and mouse rpS3.

In some embodiments, a DNA element capable of opening chromatin and/or maintaining chromatin in an open state is a naturally occurring DNA element. In other embodiments, a DNA element capable of opening chromatin and/or maintaining chromatin in an open state is artificially synthesized. In yet other embodiments, a DNA element capable of opening chromatin and/or maintaining chromatin in an open state is a combination of naturally occurring and artificially synthesized DNA elements.

In some embodiments, the nucleic acid molecule further comprises one or more of: (a) a nucleotide sequence capable of enhancing translation; (b) a nucleotide sequence capable of increasing secretion; and (c) a nucleotide sequence capable of increasing mRNA stability, operably linked to the nucleotide sequence encoding the recombinant protein.

In further embodiments, a method of producing a high titer of a recombinant protein described herein does not include a selection step.

Also encompassed by the present invention are kits for producing a high titer of a recombinant protein. In some embodiments, a kit according to the present invention comprises: a) a nucleic acid molecule comprising a DNA element capable of opening chromatin and/or maintaining chromatin in an open state operably linked to a multiple cloning site suitable for cloning a nucleotide sequence encoding the recombinant protein; and b) a reagent or device for performing high efficiency transfection (e.g., high efficiency controlled electroporation), along with instructions for use.

In some embodiments, the kit is suitable for performing controlled electroporation at a local field strength of about 250-400 V/cm.

In some embodiments, a kit according to the invention further comprises a means for monitoring the ratio between current and voltage.

In other embodiments, a kit according to the invention further comprises a cell line comprising a plurality of cells suitable for introduction of the nucleic acid molecule. In some embodiments, the plurality of cells are mammalian cells (e.g., CHO cells).

### DETAILED DESCRIPTION OF THE INVENTION

In various embodiments, the present invention provides improved methods of producing a high titer of a recombinant protein without the need for gene amplification, thereby reducing both the time as well as resources associated with the production of recombinant proteins.

In some embodiments, the present invention provides methods which employ introducing a nucleic acid molecule into a suitable cell using high efficiency transfection, where the nucleic acid molecule comprises one or more DNA elements capable of opening chromatin and/or maintaining chromatin in an open state operably linked to a nucleotide sequence encoding a recombinant protein.

In some embodiments, a method according to the present invention eliminates a selection step.

### I. DEFINITIONS

In order that the present disclosure may be more readily understood, certain terms are first defined. Additional definitions are set forth throughout the detailed description.

The terms “cell,” “cells,” “host cell,” and “host cells,” as used herein, encompass animal cells and include invertebrate, non-mammalian vertebrate and mammalian cells. Exemplary non-mammalian vertebrate cells include, for example, avian cells, reptilian cells and amphibian cells. Exemplary invertebrate cells include, but are not limited to, insect cells such as, for example, caterpillar (Spodoptera frugiperda) cells, mosquito (Aedes aegypti) cells, fruitfly (Drosophila melanogaster) cells, Schneider cells and Bombyx mori cells. See, e.g., Luckow et al., Bio/Technology 6:47-55 (1988). The cells may be differentiated, partially differentiated or undifferentiated, e.g. stem cells, including embryonic stem cells and hematopoietic stem cells. Additionally tissue samples derived from organs or organ systems may be used according to the invention.

Exemplary mammalian cells include, for example, cells derived from human, non-human primate, cat, dog, sheep, goat, cow, horse, pig, rabbit, rodents including mouse, hamster, rat and guinea pig and include, but are not limited to, BHK21 cells, CHO cells, NSO cells, Sp2/o cells, and any derivatives and progenies thereof.

Additionally, hybridoma cells can also be used in the methods of the invention. The term “hybridoma” refers to a hybrid cell line produced by the fusion of an immortal cell line of immunologic origin and an antibody producing cell. The term encompasses progeny of heterohybrid myeloma fusions, which are the result of a fusion with human cells and a murine myeloma cell line subsequently fused with a plasma cell, commonly known as a trioma cell line. Furthermore, the term is meant to include any immortalized hybrid cell line which produces antibodies such as, for example, quadromas. See, e.g., Milstein et al., Nature, 537:3053 (1983). The hybrid cell lines can be of any species, including human and mouse.

In some embodiments, a cell line used in the methods of the invention is an antibody-producing cell line. Antibody-producing cell lines may be selected and cultured using techniques well known to the skilled artisan. See, e.g., Current Protocols in Immunology, Coligan et al., Eds., Green Publishing Associates and Wiley-Interscience, John Wiley and Sons, New York (1991) which is herein incorporated by reference in its entirety, including supplements.

In general, any cell suitable for recombinant protein expression in cell culture can be used in the methods of the invention.

In some embodiments, the cells used in the methods of the present invention may include a heterologous nucleic acid molecule which encodes a desired recombinant protein, e.g., a therapeutic protein or antibody which is desired to be produced using the methods of the invention. In a particular embodiment, the methods of the present invention are useful for producing high titers of a desired recombinant protein, e.g., a therapeutic protein or antibody without the need for gene amplification.

The term “cell culture,” refers to cells grown in suspension, roller bottles, flasks and the like. Large scale approaches, such as bioreactors, including adherent cells growing attached to microcarriers in stirred fermentors, are also encompassed by the term “cell culture.” Moreover, it is possible to not only to culture contact-dependent cells, but also to use the suspension culture techniques in the methods of the claimed invention. Exemplary microcarriers include, for example, dextran, collagen, plastic, gelatin and cellulose and others as described in Butler, Spier & Griffiths, Animal cell Biotechnology 3:283-303 (1988). Porous carriers, such as, for example, Cytoline® or Cytopore®, as well as dextran-based carriers, such as DEAE-dextran (Cytodex 1®, quaternary amine-coated dextran (Cytodex 2®) or gelatin-based carriers, such as gelatin-coated dextran (Cytodex 3®) may also be used. Cell culture procedures for both large and small-scale production of proteins are encompassed by the present invention. Procedures including, but not limited to, a fluidized bed bioreactor, hollow fiber bioreactor, roller bottle culture, or stirred tank bioreactor system may be used, with or without microcarriers, and operated alternatively in a batch, fed-batch, or perfusion mode.

The terms “cell culture medium,” and “culture medium” refer to a nutrient solution used for growing animal cells, e.g., mammalian cells. Such a nutrient solution generally includes various factors necessary for cell attachment, growth, and maintenance of the cellular environment. For example, a typical nutrient solution may include a basal media formulation, various supplements depending on the cell type and, occasionally, antibiotics. In some embodiments, a nutrient solution may include at least one component from one or more of the following categories: 1) an energy source, usually in the form of a carbohydrate such as glucose; 2) all essential amino acids, and usually the basic set of twenty amino acids plus cystine; 3) vitamins and/or other organic compounds required at low concentrations; 4) free fatty acids; and 5) trace elements, where trace elements are defined as inorganic compounds or naturally occurring elements that are typically required at very low concentrations, usually in the micromolar range. The nutrient solution may optionally be supplemented with one or more components from any of the following categories: 1) hormones and other growth factors as, for example, insulin, transferrin, and epidermal growth factor; 2) salts and buffers as, for example, calcium, magnesium, and phosphate; 3) nucleosides and bases such as, for example, adenosine and thymidine, hypoxanthine; and 4) protein and tissue hydrolysates. In general, any suitable cell culture medium may be used. The medium may be comprised of serum, e.g. fetal bovine serum, calf serum or the like. Alternatively, the medium may be serum free, animal free, or protein free.

The terms “operably linked” and “operatively linked,” as used interchangeably herein, refer to the positioning of two or more nucleotide sequences or DNA elements in a manner which permits them to function in their intended manner. In some embodiments, a nucleic acid molecule according to the invention includes one or more DNA elements capable of opening chromatin and/or maintaining chromatin in an open state operably linked to a nucleotide sequence encoding a recombinant protein. In still other embodiments, a nucleic acid molecule may additionally include one or more nucleotide sequences chosen from: (a) a nucleotide sequence capable of increasing translation; (b) a nucleotide sequence capable of increasing secretion of the recombinant protein outside a cell; and (c) a nucleotide sequence capable of increasing the mRNA stability, where such nucleotide sequences are operably linked to a nucleotide sequence encoding a recombinant protein. Generally, but not necessarily, the nucleotide sequences that are operably linked are contiguous and, where necessary, in reading frame. However, although an operably linked DNA element capable of opening chromatin and/or maintaining chromatin in an open state is generally located upstream of a nucleotide sequence encoding a recombinant protein, it is not necessarily contiguous with it. Operable linking of various nucleotide sequences and/or DNA elements is accomplished by recombinant methods well known in the art, e.g. using PCR methodology, by ligation at suitable restrictions sites or by annealing. Synthetic oligonucleotide linkers or adaptors can be used in accord with conventional practice if suitable restriction sites are not present.

The term “expression” as used herein refers to transcription and/or translation of a nucleotide sequence within a host cell. The level of expression of a desired product in a host cell may be determined on the basis of either the amount of corresponding mRNA that is present in the cell, or the amount of the desired polypeptide encoded by the selected sequence. For example, mRNA transcribed from a selected sequence can be quantitated by Northern blot hybridization, ribonuclease RNA protection, in situ hybridization to cellular RNA or by PCR. Proteins encoded by a selected sequence can be quantitated by various methods including, but not limited to, e.g., ELISA, Western blotting, radioimmunoassays, immunoprecipitation, assaying for the biological activity of the protein, or by immunostaining of the protein followed by FACS analysis.

In some embodiments, the methods of the invention are capable of achieving a high titer of a recombinant protein, e.g., a therapeutic protein or an antibody.

The term “titer,” as used herein, refers to the amount of a recombinant protein produced using the methods of the invention. The amount of recombinant protein produced may be measured either at the mRNA level or at the polypeptide level, using one or more techniques well known the in art and those described herein. The term “high titer” refers to an increased amount of the recombinant protein produced using the methods of the invention, where such amount is greater than the amount obtained without the use of one or both of: (a) a DNA element capable of opening chromatin and/or maintaining chromatin in an open state; and (b) high efficiency transfection, as described herein. In some embodiments, the titer (i.e., high titer) of the recombinant protein obtained using the methods of the invention is at least about 50 mg/ml to about 100 mg/ml, about 100 mg/ml to about 200 mg/ml, about 200 mg/ml to about 300 mg/ml, about 300 mg/ml to about 400 mg/ml, about 400 mg/ml to about 500 mg/ml, about 500 mg/ml to about 600 mg/ml, about 600 mg/ml to about 700 mg/ml, about 700 mg/ml to about 800 mg/ml and about 800 mg/ml to about 900 mg/ml. In a particular embodiment, the recombinant protein is expressed at a titer of greater than about 900 mg/ml.

The term “high efficiency transfection” refers to any means of transferring a nucleic acid molecule into a cell (e.g., mammalian cell), which results in the introduction of the nucleic acid molecule into at least 50% or more, at least 60% or more, at least 70% or more, at least 80% or more, at least 90% or more, at least 95% or more, or at least 99% or more of the cells. In a particular embodiment, a nucleic acid molecule is transferred into at least 70% of the cells being transfected. In another embodiment, a nucleic acid molecule is transferred into at least 80% of the cells being transfected. Exemplary transfection methods include controlled electroporation and the use of nanoparticles including magnetic nanoparticles.

The term “electroporation,” as used herein, refers to a technique that is used for introducing chemical species (e.g., nucleic acid molecules) into biological cells, and is performed by exposing the cells to an electric potential that traverses the cell membrane. It is believed that electroporation might involve the breakdown of the cell membrane lipid bilayer leading to the formation of transient or permanent pores in the membrane that permit the chemical species to enter the cell by diffusion. Controlled electroporation is based upon the discovery that the onset and extent of electroporation in a biological cell can be correlated to changes in the electrical impedance (which as used herein means the ratio of current to voltage) of the biological cell or of a conductive medium that includes the biological cell. An increase in the current-to-voltage ratio across a biological cell occurs when the cell membrane becomes permeable due to pore formation. Likewise, a decrease in the current-to-voltage ratio through a flowing conductive fluid occurs when the fluid draws a biological cell into the region between the electrodes in a flow-through electric cell. Thus, by monitoring the impedance of the biological cell or of an electrolyte solution in which the cell is suspended, one can detect the point in time in which pore formation in the cell membrane occurs, as well as the relative degree of cell membrane permeability due to the pore formation. This information can then be used to establish that a given cell has in fact undergone electroporation, or to control the electroporation process by governing the selection of the voltage magnitude. Controlled electroporation is useful in the simultaneous electroporation of a plurality of cells, since it provides a direct indication of the actual occurrence of electroporation and an indication of the degree of electroporation averaged over the cells. The method is likewise useful in the electroporation of biological tissue (masses of biological cells with contiguous membranes) for the same reasons. By “high efficiency controlled electroporation,” it is meant that the chemical species being introduced using controlled electroporation (e.g., a nucleic acid molecule) enters at least 70% of the cells, or at least 75% of the cells, or at least 80% of the cells, or at least 90% of the cells, or at least 95% of the cells, or more, that are being electroporated. High efficiency controlled electroporation can be performed using known methods in the art and those described herein. In one aspect, high efficiency controlled electroporation is performed using an electroporation device which includes a barrier. In another aspect, high efficiency controlled electroporation is performed using an electroporation device which includes one or more capillaries. The onset of electroporation as well as the percentage of cells that are electroporated can be measured using well known assays in the art, e.g., by assaying for internalization of membrane-impermeant molecules such as, e.g., SYTOX green.

Other exemplary methods of achieving high efficiency transfection include, e.g., nanoparticle transfection (See Sang et al., Biochimica et biophysics acta (2007), vol. 1770, no. 5: 747-752 and reagents sold by SIGMA-ALDRICH) and the use of magnetic nanoparticles (e.g., CombiMag sold by OZ Biosciences). Such methods can be used alone or in combination with additional transfection reagents or devices known in the art and those described herein.

The term “recombinant protein” or “recombinant polypeptide” produced by the methods of the invention generally refers to a peptide or protein, typically more than about ten amino acids in length produced by cells in culture using methods of the invention. A polypeptide produced by the methods of the invention is typically exogenous, i.e., heterologous or foreign, to the cells producing the polypeptide. Exemplary polypeptides produced by cells in culture using methods of the present invention include therapeutic proteins and antibodies and antigen binding fragments thereof. Also encompassed by the present invention are fusion proteins.

The term “immunoglobulin” or “antibody” (used interchangeably herein) refers to a protein having a basic four-polypeptide chain structure consisting of two heavy and two light chains, said chains being stabilized, for example, by interchain disulfide bonds, which has the ability to specifically bind antigen. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains). Immunoglobulins or antibodies may be monoclonal or polyclonal and may exist in monomeric or polymeric form, for example, IgM antibodies which exist in pentameric form and/or IgA antibodies which exist in monomeric, dimeric or multimeric form. The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc and/or Fv fragments.

The term “antigen-binding fragment” refers to a polypeptide portion of an immunoglobulin or antibody that binds an antigen or competes with intact antibody (i.e., with the intact antibody from which they were derived) for antigen binding (i.e., specific binding). Binding fragments can be produced by recombinant DNA techniques, or by enzymatic or chemical cleavage of intact immunoglobulins. Binding fragments include Fab, Fab′, F(ab′)₂, Fabc, Fv, single chains, and single-chain antibodies.

The terms “polynucleotide” and “nucleic acid molecule,” used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. A nucleic acid molecule can take many different forms, e.g., a gene or gene fragment, one or more exons, one or more introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. As used herein, “DNA” or “nucleotide sequence” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides. In a particular embodiment, a nucleic acid molecule comprises a nucleotide sequence encoding a recombinant protein such as, for example, a therapeutic protein or an antibody, operably linked to a DNA element capable of opening chromatin and/or maintaining chromatin in an open state.

The term “a DNA element capable of opening chromatin and/or maintaining chromatin in an open state” refers to any DNA sequence or element which has the ability to make chromatin more accessible to transcription factors and facilitate reproducible expression of an operably-linked gene, where such a DNA sequence or element is not derived from a locus control region. Open chromatin or chromatin in an open state refers to chromatin in a de-condensed state and is also referred to as euchromatin. Condensed chromatin is also referred to as heterochromatin. Chromatin in a closed (condensed) state is transcriptionally silent. Whereas, chromatin in an open (de-condensed) state is transcriptionally competent. The establishment of an open chromatin structure is characterized by DNase I sensitivity, DNA hypomethylation and histone hyperacetylation. Standard methods for identifying open chromatin are well known to those skilled in the art and are described in Wu, 1989, Meth. Enzymol., 170, 269-289; Crane-Robinson et al., 1997, Methods, 12, 48-56; Rein et al., 1998, N. A. R, 26, 2255-2264.

A “locus control region” (LCR) refers to a genetic element which is obtained from a tissue-specific locus of a eukaryotic host cell and which, when linked to a gene of interest and integrated into a chromosome of a host cell, confers tissue-specific, integration site-independent, copy number-dependent expression on the gene of interest.

Reproducible expression means that the DNA element when operably-linked to a gene of interest gives substantially the same level of expression of the operably-linked gene over an extended period of time irrespective of its chromatin environment and irrespective of the cell type. In some embodiments, substantially the same level of expression means a level of expression which has a standard deviation from an average value of less than 48%, or less than 40%, or less than 25% on a per-gene-copy basis. Alternatively, substantially the same level of expression means that the level of expression varies by less than 10 fold, less than 5 fold, or less than 3 fold on a per gene copy basis. In some embodiments, a DNA element capable of opening chromatin and/or maintaining chromatin in an open state increases the expression of an operably-linked gene by at least 2 fold, or at least 3 fold, or at least 4 fold, or at least 5 fold, or at least 10 fold, or at least 20 fold, or at least 30 fold, or at least 40 fold, or at least 50 fold, or at least 60 fold, or at least 70 fold, or at least 80 fold, or at least 90 fold, or at least 95 fold, or at least 100 fold, or at least 150 fold, or at least 200 fold, or more, relative to the expression without such an operably-linked DNA element. In some embodiments, a DNA element capable of opening chromatin and/or maintaining chromatin in an open state obtains a reproducible expression of an operably-linked gene over an extended period of time. For example, in some embodiments, an operably-linked gene is expressed at substantially the same level over a period of at least 5 days, 10 days, or at least 15 days, or at least 20 days, or at least 30 days, or at least 40 days, or at least 45 days, or at least 60 days, or at least 70 days, or at least 80 days, at least 90 days or more, relative to the expression level when the gene is not operably-linked to a DNA element capable of opening chromatin and/or maintaining chromatin in an open state. In other embodiments, an operably-linked gene is expressed at higher levels over an extended period of time relative to the levels when the gene is not operably-linked to a DNA element capable of opening chromatin and/or maintaining chromatin in an open state. Exemplary DNA elements capable of opening chromatin and/or maintaining chromatin in an open state include, but are not limited to, extended methylation-free CpG islands derived from the promoter regions of ubiquitously expressed genes (UCOEs), matrix and/or scaffolding attachment regions (MARs) and stabilizing and antirepressor regions (STARs). One skilled in the art can readily identify such DNA elements using well known assays in the art and those described herein.

In some embodiments, a DNA element capable of opening chromatin and/or maintaining chromatin in an open state is a naturally occurring DNA element. By naturally occurring DNA element, it is meant that the DNA element occurs in nature, e.g., it is isolated from the promoter region of a ubiquitously expressed gene, and its sequence is not altered from the naturally occurring sequence.

In other embodiments, a DNA element capable of opening chromatin and/or maintaining chromatin in an open state is an artificially synthesized DNA element. By artificially synthesized, it is meant that the DNA element does not occur in nature, e.g., a DNA element isolated from the promoter region of a ubiquitously expressed gene which is combined with a second DNA element isolated from the promoter region of another ubiquitously expressed gene, thereby resulting in an artificial construct, as the two elements do not normally occur together in nature. Alternatively, a DNA element may be modified in sequence using various techniques well known in the art from its naturally occurring sequence, thereby resulting in a DNA element that does not normally occur in nature.

In yet another embodiment, a DNA element capable of opening chromatin and/or maintaining chromatin in an open state is a combination of naturally occurring and artificially synthesized DNA elements.

The term “methylation-free CpG island” refers to CpG-islands have an average GC content of approximately 60%, compared with a 40% average in bulk DNA. One skilled in the art can easily identify CpG-islands using standard techniques such as restriction enzymes specific for C and G sequences, which are well known in the art. Exemplary methods for the identification of CpG islands can be found in, e.g., Gardiner-Garden et al., J. Mol. Biol. 1987,196:261-82, incorporated by reference herein, and using computer programs such as CpGplot which are readily available to one of ordinary skill in the art for analyzing and identifying CpG islands (e.g., http://www.ebi.ac.uk/emboss/cpgplot/) and Grailexp (http://compbio.ornl.gov/grailexp).

The term “an extended methylation-free CpG island,” as used herein, refers to a methylation-free CpG island which is at least 300 bp, or at least 500 bp, or at least 1000 bp, or at least 1500 bp, or at least 2000 bp, or at least 2500 bp, or at least 3000 bp in length and is derived from the promoter region of a ubiquitously expressed gene. Such islands are well known in the art and are described in detail in U.S. Pat. Nos. 6,964,951; 6,689,606; 6,881,556; and 6,949,361 and PCT Application Publication No. WO 2004/067701, each of which is incorporated by reference herein in their entirety.

In some embodiments, an extended methylation-free CpG island includes one or more transcription factor binding sites. In other embodiments, an extended methylation-free CpG island includes a promoter and/or enhancer sequence. In yet other embodiments, an extended methylation-free CpG island includes a dual or bi-directional promoter. Although an extended methylation-free CpG island may include a promoter, as used herein, such islands are typically used in conjunction with one or more heterologous promoters which are not typically associated with the island, e.g., human or guinea pig CMV promoter. In some embodiments, a heterologous promoter replaces the endogenous promoter found within the CpG island.

Extended methylation-free CpG islands can be defined, e.g., by identifying the borders of such islands. For example, the borders of the extended methylation-free CpG islands can be defined through the use of PCR in combination with restriction endonuclease enzymes whose ability to digest (cut) DNA at their recognition sequence is sensitive to the methylation status of any CpG residues that are present. One such enzyme is HpaII, which recognizes and digests at the site CCGG, which is commonly found within CpG islands, but only if the central CG residues are not methylated. Therefore, PCR conducted with HpaII-digested DNA and over a region harboring HpaII sites, does not give an amplification product due to HpaII digestion if the DNA is unmethylated. The PCR will only give an amplified product if the DNA is methylated. Therefore, beyond the methylation-free region, HpaII will not digest the DNA a PCR amplified product will be observed thereby defining the boundaries of the “extended methylation-free CpG island.”

Exemplary extended methylation-free CpG islands include, but are not limited to, those derived from the promoter regions of the human RNPA2 gene (SEQ ID NOs:2, 3 and 4), RPS3 gene (Accession No. NM012052; SEQ ID NO:1), RPL4 gene (Accession No. NT_{—}039474), RPL5 gene (NT_{—}039308), RPL10a gene (Accession No. NT_{—}039649), RPL13a gene (Accession No. NT_{—}039420), RPL19 gene (Accession No. NT_{—}039521), RPL24 gene (Accession No. NT_{—}096987), RPL27a gene (Accession No. NT_{—}039433), Terf2ip gene (Accession No. AB041557), human glyceryldehyde-3 phosphate dehydrogenase gene (Accession No. M32599), tubulin alpha-1 chain gene (Accession No. M13445), and RPS11 gene (Accession No. AK011207). Additional examples of ubiquitously expressed or housekeeping genes can be found in, e.g., Trends in Genetics 19, 362-365 (2003), incorporated by reference herein.

The term “matrix attachment region,” or “scaffold attachment region,” or “scaffold/matrix attachment region,” or “MAR” or “S/MAR,” as used interchangeably herein, refers to a DNA element which is capable of binding isolated nuclear scaffolds or nuclear matrices in vitro with high affinity. (See, e.g., Hart and Laemmli (1988) Curr. Opin. Genet. Dev., 8:519-525). It has been reported that MAR DNA elements can increase expression of a heterologous gene in cell culture. (See, e.g., Kalos and Fournier (1995) Mol. Cell Biol. 15:198-207; Phi-Van et al. (1990) Mol. Cell Biol. 10:2302-2307; Klehr et al. (1991) Biochemistry 30:1264-1270; and Poljak et al. (1994) Nuc. Acid Res. 22:4386-4394). Exemplary MAR DNA elements can be found in, for example, U.S. Pat. No. 7,129,062, incorporated by reference herein in its entirety. In a particular embodiment, a MAR element used in the methods of the invention is a chicken lysozyme MAR element, as set forth in U.S. Pat. No. 7,129,062, and functional fragments thereof. One skilled in the art can readily identify MAR elements based on the well known assays in the art coupled with those described herein, e.g., those described in Mesner et al. (2003) Proc. Natl. Acad. Sci., 3281-3286 and Weber et al., Mol Cell Biol. (2003) December; 23(24): 8953-8959. In another embodiment, a MAR DNA element used in the methods of the invention is a human β-globin MAR element. Exemplary MAR DNA elements which may be used in the methods of the invention are set forth in SEQ ID NOs:11-14.

The term “stabilizing and antirepressor region” or “STAR” refers to a DNA element which has the ability to block heterochromatin-mediated transgene expression. STAR DNA elements can be readily identified using known techniques for assaying for gene transcription modulating properties of DNA elements, e.g., those described in WO03/004704, WO 2004/056986 and EP01202581.3, incorporated by reference herein in their entirety. Non-limiting examples of STAR sequences which may be used in the methods of invention include sequence set forth in SEQ. ID. NOs. 1-66 in US Patent Publication No. 20060141577.

The term “a nucleotide sequence capable of increasing translation” refers to a nucleotide sequence which is capable of increasing the synthesis of a polypeptide from an mRNA. An increase in synthesis of the polypeptide can either be an increase in the overall amount of the polypeptide produced or an increase in the rate of synthesis of the polypeptide. In one embodiment, a nucleotide sequence capable of increasing translation is operably linked to a nucleotide sequence encoding a recombinant protein. The ability of the nucleotide sequence capable of increasing translation and be measured by assaying for an increase in the amount of the recombinant protein produced in the presence of the nucleotide sequence capable of increasing translation or by the rate of synthesis of the recombinant protein over time.

The term “a nucleotide sequence capable of increasing secretion” refers to a nucleotide sequence, which when operably linked to a nucleotide sequence encoding a protein, has the ability to promote secretion of the protein outside the cell. Typically, such a nucleotide sequence comprises an appropriate native or heterologous signal peptide (leader sequence). The choice of signal peptide or leader depends on the type of host cells in which the recombinant protein is to be produced, and a heterologous signal peptide can replace the native signal sequence. Exemplary sequences which may be used in the methods of the invention include, for example, a signal peptide derived from a luciferase gene from Gaussia princeps (Genbank Accession No. AY015993). Nucleotide sequences which are capable of increasing secretion, also referred to as signal peptide sequences, can be identified using software programs well known in the art, such as, for example, SignalP Server (http://www.cbs.dtu.dk/services/SignalP).

The term “a nucleotide sequence capable of increasing mRNA stability,” as used herein, refers to a nucleotide sequence, which when operably linked to a nucleotide sequence encoding a recombinant protein, increases the half-life of the mRNA which is translated into the recombinant protein. Typically, such nucleotide sequences are derived from the 3′ or 5′ untranslated regions (or UTRs) of genes.

### II. EXEMPLARY CELLS

Without wishing to be bound by theory, it is contemplated that any cell line which is capable of producing a recombinant protein may be used in the methods of the invention. In a particular embodiment, cells used in the methods of the invention are transfected with a nucleic acid molecule comprising a nucleotide sequence encoding a recombinant polypeptide, e.g., a therapeutic protein or an antibody. In a particular embodiment, the cells used in the methods of the invention are eukaryotic cells, e.g., mammalian cells. Examples of mammalian cells include, but are not limited to, for example, monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); MRC 5 cells; FS4 cells; NSO mouse myeloma cells (ECACC; SIGMA), and a human hepatoma line (Hep G2). Additional examples of useful cell lines include, but are not limited to, HT1080 cells (ATCC CCL 121), MCF-7 breast cancer cells (ATCC BTH 22), K-562 leukemia cells (ATCC CCL 243), KB carcinoma cells (ATCC CCL 17), 2780AD ovarian carcinoma cells (see Van der Blick, A. M. et al., Cancer Res. 48:5927-5932 (1988), Raji cells (ATCC CCL 86), Jurkat cells (ATCC TIB 152), Namalwa cells (ATCC CRL 1432), HL-60 cells (ATCC CCL 240), Daudi cells (ATCC CCL 213), RPMI 8226 cells (ATCC CCL 155), U-937 cells (ATCC CRL 1593), Bowes Melanoma cells (ATCC CRL 9607), WI-38VA13 subline 2R4 cells (ATCC CLL 75.1), and MOLT-4 cells (ATCC CRL 1582), as well as heterohybridoma cells produced by fusion of human cells and cells of another species. These and other cells and cell lines are available commercially, for example from the American Type Culture Collection (Virginia, USA). Many other cell lines are known in the art and will be familiar to the ordinarily skilled artisan; such cell lines therefore can be used equally well in the methods of the present invention. In a particular embodiment, cells used in the methods of the invention are CHO cells or NSO cells.

Hybridomas and antibody-producing cells may also be used in the methods of the invention.

### III. EXEMPLARY NUCLEOTIDE SEQUENCES AND VECTORS

In some embodiments, a nucleic acid molecule comprising a nucleotide sequence encoding a recombinant protein of interest is introduced into a host cell using high efficiency transfection, as described herein, where the nucleotide sequence is operably linked to a DNA element capable of opening chromatin and/or maintaining chromatin in an open state. For example, a first nucleic acid molecule comprising a nucleotide sequence encoding a desired recombinant protein of interest is cloned into a suitable expression vector, which includes the nucleotide sequence encoding the recombinant protein operably linked to a DNA element capable of opening chromatin and/or maintaining chromatin in an open state.

Any suitable vector may be used according to the invention. Nucleotide sequences can be stably integrated into the host cell genome using, for example, retroviral (Miller, 1992, Curr. Top. Microbiol. Immunol 158:1; Miller et al., 1993, Meth. Enzymol. 217: 581) or adeno-associated viral (MV) vectors (Muzyczka, 1992, Curr. Top. Microbiol. Immunol., 158: 97; Flotte and Carter, 1995, Gene Ther. 2: 357). Alternatively, nucleotide sequences encoding proteins can be incorporated within self-replicating episomal vectors comprising viral origins of replication such as those from EBV (Yates et al., 1985, Nature 313: 812), human papovavirus BK (De Benedetti and Rhoads, 1991, Nucl. Acids Res., 19: 1925; Cooper and Miron, 1993, Hum. Gene Ther. 4: 557; and BPV-1 (Piirsoo et al., 1996, EMBO J. 15:1).

Vectors and methods for genetically engineering cells and/or cell lines to express a protein of interest are well known to those skilled in the art; for example, various techniques are illustrated in Current Protocols in Molecular Biology, Ausubel et al., eds. (Wiley & Sons, New York, 1988, and quarterly updates); Sambrook et al., Molecular Cloning: A Laboratory Manual (Cold Spring Laboratory Press, 1989) and Kaufman, R. J., Large Scale Mammalian Cell Culture (1990, pp. 15-69).

Additional regulatory sequences may also be included in the expression vectors described herein. These may be derived from mammalian, microbial, viral, and/or insect genes. Examples of regulatory sequences include transcriptional promoters, operators, enhancers, ribosome binding sites (see e.g. Kozak (1991), J. Biol. Chem. 266:19867-70), sequences that can control transcriptional and translational termination, and polyadenylation signals (see e.g. McLauchlan et al. (1988), Nucleic Acids Res. 16:5323-33).

Some commonly used promoter and enhancer sequences are derived from viral genomes, for example polyoma virus, adenovirus 2, simian virus 40 (SV40), and human cytomegalovirus. For example, the human CMV promoter/enhancer of immediate early gene 1 may be used (see, e.g., Patterson et al. (1994), Applied Microbiol. Biotechnol. 40:691-98). DNA sequences derived from the SV40 viral genome, for example, SV40 origin, early and late promoter, enhancer, splice, and polyadenylation sites can be used to provide other genetic elements for expression of a polypeptide in a eukaryotic host cell. Viral early and late promoters are particularly useful because both are easily obtained from a viral genome as a fragment, which can optionally also contain a viral origin of replication (Fiers et al. (1978), Nature 273:113; Kaufman (1990), Meth. in Enzymol. 185:487-511). Smaller or larger SV40 fragments can also be used. In some embodiments, expression vectors used in the methods of the invention include a human or a guinea pig CMV promoter.

In some embodiments, a nucleotide sequence encoding a recombinant protein is operably-linked to one or more nucleotide sequences chosen from: (a) a nucleotide sequence capable of increasing translation; (b) a nucleotide sequence capable of increasing secretion; and (c) a nucleotide sequence capable of increasing mRNA stability.

Additional control sequences shown to improve expression of heterologous genes from mammalian expression vectors include such elements as the expression augmenting sequence element (EASE) derived from CHO cells (Morris et al., Animal Cell Technology, pp. 529-534 (1997); U.S. Pat. No. 6,312,951 B I; U.S. Pat. No. 6,027,915; U.S. Pat. No. 6,309,841 B 1) and the tripartite leader (TPL) and VA gene RNAs from Adenovirus 2 (Gingeras et al. (1982), J. Biol. Chem. 257:13475-13491) and internal ribosome entry site (IRES) sequences that allow mRNAs to be translated efficiently.

A gene encoding a selectable marker is often used to facilitate the identification of recombinant cells. Selection of transformants can be performed using methods such as, for example, the dihydrofolate reductase (DHTR) selection scheme or resistance to cytotoxic drugs (see, e.g., Kaufman et al. (1990), Meth. in Enzymology 185:487-511). A suitable cell line for DHFR selection can be, for example, CHO line DX-B 11, which is deficient in DHFR (see, e.g., Urlaub and Chasin (1980), Proc. Natl Acad. Sci. USA 77:4216-4220). Other examples of selectable markers include those conferring resistance to antibiotics, such as G418 and hygromycin B.

In certain embodiments of the invention, a gene encoding for a selectable marker is not necessary due to the use of the combination of a DNA element capable of opening chromatin and/or maintaining chromatin in an open state and high efficiency transfection, as pools of cells expressing a recombinant protein can be used instead of selecting a particular clone.

In some embodiments, an exogenous nucleic acid which is used for producing a protein by the methods according to the invention is isolated from a cDNA library or a genomic library. For example, in order to isolate a nucleic acid encoding a protein of interest, a cDNA library may be screened with probes designed to identify the gene or a cDNA clone encoding the protein. For cDNA expression libraries, suitable probes include monoclonal or polyclonal antibodies that recognize and specifically bind to the protein of interest; oligonucleotides of about 20-80 bases in length that encode known or suspected portions of the protein from the same or different species; and/or complementary or homologous cDNAs or fragments thereof for the same or a similar gene. Appropriate probes for screening genomic DNA libraries include, but are not limited to, oligonucleotides, cDNAs, or fragments thereof that encode the same or a similar gene, and/or homologous genomic DNAs or fragments thereof. Screening the cDNA or genomic library with the selected probe may be conducted using standard procedures as described in chapters 10-12 of Sambrook et al., Molecular Cloning: A Laboratory Manual (New York: Cold Spring Harbor Laboratory Press, 1989).

In various embodiments described herein, particular sequences described herein as well as homologs and fragments of such sequences can be used in the methods of the invention, so long as they have the desired activity. For example, in some embodiments, sequences that are at least 70% identical, or at least 80% identical, or at least 90% identical, or at least 95% or more identical, to particular sequences encompassed by the present invention are useful in the methods of the invention.

### IV. EXEMPLARY RECOMBINANT PROTEINS

The methods of the invention can be used to produce any desired recombinant protein or fragment thereof. In some embodiments, a recombinant protein produced using the methods described herein is a therapeutic protein. In other embodiments, the recombinant protein is an antibody or functional fragment thereof. Antibodies which may be produced using the methods of the invention include, for example, polyclonal, monoclonal, monospecific, polyspecific, fully human, humanized, single-chain, chimeric, hybrid, mutated, and CDR-grafted antibodies, and antigen-binding fragments thereof, such as, for example, Fab, F(ab′)₂, Fv, and scFv. The antibodies can be specific for any desirable antigen comprising a suitable epitope. Desirable antigens may include for example, a marker found in or associated with a mammalian cell, a marker associated with a tumor or a marker associated with a disease or condition. Examples of tumor markers include tumor antigen CA 125, tumor antigen gp72 LCG (which is a gene product that is expressed in association with lung cancer), HER-2, a tumor-associated glycoprotein, and tumor antigen MUC 1. Other markers for cancer include hTERT (Ferber et al. 2003, Oncogene 22:3813), Ki-67 (Kruse et al. 2002, Am. J. Surg. Pathol., 26:1501), cyclin E (Yasmeen et al. 2003, Expert Rev. Mol. Diagn. 3(5):617) and histone H3 (Rakowicz-Szulczynska, et al. 1996, Cancer Biother. Radiopharm. 11:77).

In some embodiments, methods of the invention are used for producing high titers of antibodies or antigen-binding fragments thereof. An antibody may be specific to a cell surface protein such as a growth factor or hormone receptor.

Antibodies within the scope of the present invention include, but are not limited to: anti-HER2 antibodies including Trastuzumab (HERCEPTIN®) (Carter et al., Proc. Natl. Acad. Sci. USA, 89:4285-4289 (1992), U.S. Pat. No. 5,725,856); anti-CD20 antibodies such as chimeric anti-CD20 “C2B8” as in U.S. Pat. No. 5,736,137 (RITUXAN®), a chimeric or humanized variant of the 2H7 antibody as in U.S. Pat. No. 5,721,108, B1, or Tositumomab (BEXXAR®); anti-IL-8 (St John et al., Chest, 103:932 (1993), and International Publication No. WO 95/23865); anti-VEGF antibodies including humanized and/or affinity matured anti-VEGF antibodies such as the humanized anti-VEGF antibody huA4.6.1 AVASTIN®. (Kim et al., Growth Factors, 7:53-64 (1992), International Publication No. WO 96/30046, and WO 98/45331, published Oct. 15, 1998); anti-PSCA antibodies (WO01/40309); anti-CD40 antibodies, including S2C6 and humanized variants thereof (WO00/75348); anti-CD11a (U.S. Pat. No. 5,622,700, WO 98/23761, Steppe et al., Transplant Intl. 4:3-7 (1991), and Hourmant et al., Transplantation 58:377-380 (1994)); anti-IgE (Presta et al., J. Immunol. 151:2623-2632 (1993), and International Publication No. WO 95/19181); anti-CD18 (U.S. Pat. No. 5,622,700, issued Apr. 22, 1997, or as in WO 97/26912, published Jul. 31, 1997); anti-IgE (including E25, E26 and E27; U.S. Pat. No. 5,714,338, issued Feb. 3, 1998 or U.S. Pat. No. 5,091,313, issued Feb. 25, 1992, WO 93/04173 published Mar. 4, 1993, or International Application No. PCT/US98/13410 filed Jun. 30, 1998, U.S. Pat. No. 5,714,338); anti-Apo-2 receptor antibody (WO 98/51793 published Nov. 19, 1998); anti-TNF-α antibodies including cA2 (REMICADE®), CDP571 and MAK-195 (See, U.S. Pat. No. 5,672,347 issued Sep. 30, 1997, Lorenz et al. J. Immunol. 156(4):1646-1653 (1996), and Dhainaut et al. Crit. Care Med. 23(9):1461-1469 (1995)); anti-Tissue Factor (TF) (European Patent No. 0 420 937 B1 granted Nov. 9, 1994); anti-human α₄β₇ integrin (WO 98/06248 published Feb. 19, 1998); anti-EGFR (chimerized or humanized 225 antibody as in WO 96/40210 published Dec. 19, 1996); anti-CD3 antibodies such as OKT3 (U.S. Pat. No. 4,515,893 issued May 7, 1985); anti-CD25 or anti-tac antibodies such as CHI-621 (SIMULECT®) and (ZENAPAX®) (See U.S. Pat. No. 5,693,762 issued Dec. 2, 1997); anti-CD4 antibodies such as the cM-7412 antibody (Choy et al. Arthritis Rheum 39(1):52-56 (1996)); anti-CD52 antibodies such as CAMPATH-1H (Riechmann et al. Nature 332:323-337 (1988)); anti-Fc receptor antibodies such as the M22 antibody directed against FcγRI as in Graziano et al. J. Immunol. 155(10):4996-5002 (1995); anti-carcinoembryonic antigen (CEA) antibodies such as hMN-14 (Sharkey et al. Cancer Res. 55(23Suppl): 5935s-5945s (1995); antibodies directed against breast epithelial cells including huBrE-3, hu-Mc 3 and CHL6 (Ceriani et al. Cancer Res. 55(23): 5852s-5856s (1995); and Richman et al. Cancer Res. 55(23 Supp): 5916s-5920s (1995)); antibodies that bind to colon carcinoma cells such as C242 (Litton et al. Eur J. Immunol. 26(1):1-9 (1996)); anti-CD38 antibodies, e.g. AT 13/5 (Ellis et al. J Immunol. 155(2):925-937 (1995)); anti-CD33 antibodies such as Hu M195 (Jurcic et al. Cancer Res 55(23 Suppl):5908s-5910s (1995) and CMA-676 or CDP771; anti-CD22 antibodies such as LL2 or LymphoCide (Juweid et al. Cancer Res 55(23 Suppl):5899s-5907s (1995)); anti-EpCAM antibodies such as 17-1A (PANOREX®); anti-GpIIb/IIIa antibodies such as abciximab or c7E3 Fab (REOPRO®); anti-RSV antibodies such as MEDI-493 (SYNAGIS®); anti-CMV antibodies such as PROTOVIR®; anti-HIV antibodies such as PRO542; anti-hepatitis antibodies such as the anti-Hep B antibody OSTAVIR®; anti-CA 125 antibody OvaRex; anti-idiotypic GD3 epitope antibody BEC2; anti-αvβ3 antibody VITAXIN®; anti-human renal cell carcinoma antibody such as ch-G250; ING-1; anti-human 17-1A antibody (3622W94); anti-human colorectal tumor antibody (A33); anti-human melanoma antibody R24 directed against GD3 ganglioside; anti-human squamous-cell carcinoma (SF-25); and anti-human leukocyte antigen (HLA) antibodies such as Smart ID10 and the anti-HLA DR antibody Oncolym (Lym-1). The preferred target antigens for the antibody herein are: HER2 receptor, VEGF, IgE, CD20, CD11a, and CD40.

The recombinant protein may be a cellular protein such as a receptor (e.g., membrane bound or cytosolic) or a structural protein (e.g. a cytoskeletal protein). The recombinant protein may be cellular factor secreted by the cell or used internally in one or more signal transduction pathways. Non limiting examples include, but are not limited to, CD2, CD3, CD4, CD8, CD11a, CD14, CD18, CD20, CD22, CD23, CD25, CD33, CD40, CD44, CD52, CD80 (B7.1), CD86 (B7.2), CD147, IL-1, IL-2, IL-3, IL-7, IL-4, IL-5, IL-8, IL-10, IL-2 receptor, IL-4 receptor, IL-6 receptor, IL-13 receptor, IL-18 receptor subunits, PDGF, EGF receptor, VEGF receptor, hepatocyte growth factor, osteoprotegerin ligand, interferon gamma, B lymphocyte stimulator C5 complement TAG-72, integrin alpha 4 beta 7, the integrin VLA-4, B2 integrins, TRAIL receptors 1, 2, 3, and 4, RANK, RANK ligand, TNF, the adhesion molecule VAP-1, epithelial cell adhesion molecule (EpCAM), intercellular adhesion molecule-3 (ICAM-3), leukointegrin adhesin, the platelet glycoprotein gp IIb/IIIa, cardiac myosin heavy chain, parathyroid hormone, rNAPc2, and CTLA4 (which is a cytotoxic T lymphocyte-associated antigen).

The recombinant protein may also be derived from an infectious agent such as a virus, a bacteria, or fungus. For example, the protein may be derived from a viral coat or may be a viral enzyme or transcription factor. The protein may be derived from a bacterial membrane or cell wall, or may be derived from the bacterial cytosol. The protein may be a yeast enzyme, transcription factor, or structural protein. The yeast protein may be membrane bound, cytsolic, or secreted. Examples of infectious agents include, but are not limited to, respiratory syncitial virus, human immunodeficiency virus (HIV), hepatitis B virus (HBV), hepatitis C virus (HCV), Streptococcus mutans, and Staphlycoccus aureus, and Candida albicans.

The methods of the invention can also be used to produce recombinant fusion proteins comprising all or part of any of the above-mentioned proteins. For example, recombinant fusion proteins comprising one of the above-mentioned proteins plus a multimerization domain, such as a leucine zipper, a coiled coil, an Fc portion of an antibody, or a substantially similar protein, can be produced using the methods of the invention. See e.g. International Application No. WO 94/10308; Lovejoy et al. (1993), Science 259:1288-1293; Harbury et al. (1993), Science 262: 1401-05; Harbury et al. (1994), Nature 371:80-83; Hang.kansson et al. (1999), Structure 7:255-64.

Also encompassed by this invention are pharmaceutical compositions including one or more recombinant proteins produced by the methods described herein. In some embodiments, pharmaceutical compositions further include a pharmaceutically acceptable carrier. The term “pharmaceutically-acceptable carrier” as used herein means one or more compatible solid or liquid filler, diluents or encapsulating substances which are suitable for administration into a subject.

### V. HIGH EFFICIENCY TRANSFECTION METHODS

The methods of the invention employ high efficiency transfection methods and are useful for the production of high titers of recombinant proteins, e.g., therapeutic proteins and antibodies, without the need for gene amplification.

In a particular aspect, high efficiency controlled electroporation is used for introducing the various nucleic acid molecules into a host cell, using an electroporation device which includes a barrier that directs the electric current flow and hence the ion flow through a flow path that passes through the biological cell while permitting substantially no electric current to bypass the biological cell. Exemplary devices and methods for performing controlled electroporation can be found, for example, in U.S. Pat. Nos. 6,300,108; 6,562,604; 6,387,671; 6,403,348; 6,482,619; 7,053,063, each of which are incorporated by reference herein in their entirety.

In one aspect of the invention, controlled electroporation involves the use of an apparatus containing two liquid-retaining chambers separated by a barrier that is substantially impermeable to an electric current. The barrier contains an opening that is smaller than the biological cell such that the biological cell once lodged in the opening will plug or close the opening. To achieve electroporation, the biological cell is secured over the opening by mechanical or chemical means, e.g., in a reversible manner so that the biological cell can later be removed without damage to the biological cell. Once the biological cell is secured over the opening, a voltage is imposed between the two chambers and across the biological cell residing in the opening. The passage of current between the chambers is thus restricted to a path passing through the opening and hence through the biological cell. By monitoring the current-voltage relation in the electric cell, the onset of electroporation is detected and the degree of pore formation is controlled, to both assure that electroporation is occurring and to prevent excessive pore formation and cell death. The user is thus afforded a highly precise knowledge and control of the condition of and the flux across the biological cell membrane. The device may thus comprise two electrodes. The polarity of each respective electrode may be alternated back and forth thus permitting penetration of a target nucleic acid through the cell membrane from at least two distinct points. For example, the points may be approximately 180° apart in a plane of the cell.

The electroporation device may comprise an internal support to hold a single biological cell, or a plurality of biological cells, and an internal barrier that restricts the electric current flow in the device to a flow path that passes through the biological cell. The electroporation device may comprise one or more chambers suitable for holding a buffer. Where a plurality of chambers is present each chamber may hold the same buffer, or a different buffer. When no voltage is applied, the structure can be used for diffusive transport alone, unassisted by voltage-induced pore formation. The configuration of the barrier, and the two chambers in embodiments that include two chambers, is not critical to the electroporation cell, and can vary widely while still serving its purpose. Since biological cells are microscopic in size, however, the apparatus may be the size of electronic chips, fabricated by microfabrication techniques such as those used in electronic chip manufacture. The chambers may be constructed as flow-through chambers to allow the passage of the liquids in continuous flow, intermittent flow, or flow at the direction of the user, and to allow changes in the concentrations, pressure, and other conditions as needed to achieve close control over the passage of species across the biological cell membrane. The apparatus may comprise layers or platelets with appropriate openings that form flow passages when the layers or platelets are bonded together.

Flow-through chambers offer the advantage of permitting the successive entry and removal of individual cells so that large numbers of cells can be treated in succession. Flow-through chambers also permit replenishment of solute-depleted solutions so that concentration gradients can be continuously maintained when desired. A further function that can be served by flow-through chambers is the increase and decrease of pressure, a function that is useful for various purposes as described below.

The support for the biological cell in this structure can be any structure that secures the biological cell in a fixed position and that allows the passage of electric current. The most convenient support is an opening in the barrier. Securing a biological cell over the opening serves to close, seal or plug the opening, thereby directing the passage of electric current, diffusive transport, or both, through the cell and eliminating or minimizing leakage around the cell. A mechanical means of achieving this is to impose a pressure differential across the opening in a direction that will press the cell against the opening. The diameter of the opening may be smaller than that of the cell, and the cell upon entering the apparatus will pass into one of the two chambers. By increasing the pressure in the chamber in which the cell resides, or lowering the pressure in the other chamber, the cell will be forced against the opening, closing it off. Once the procedure is completed, the cell is readily released from the opening by equalizing the pressures in the two chambers or by reversing the differential such that the higher pressure is in the chamber other than the chamber in which the cell was introduced. The flow of liquid in the chamber in which the cell was introduced will then remove the cell from the opening, exposing the opening for another cell.

An alternative method of sealing the opening with the cell is by the use of a coating on the barrier surface, or over the rim of the opening, of a substance that binds to the cell membrane. Since biological cell membranes are negatively charged, the coating may be a substance that bears a positive charge, such as polylysine, polyarginine, or polyhistidine. The biological cell can be directed to the opening by a pressure differential across the opening, and held in place by the coating. Once the procedure is completed, the cell can be released from the coating by momentarily increasing the flow rate of the liquid in the chamber on the cell side of the opening, or by imposing a reverse pressure differential across the opening to urge the cell away from the opening.

In another aspect, controlled electroporation is performed using an electroporation device such as, e.g., described in Wang et al., Anal. Chem, (2006) 78:5158-5164.

In one aspect, high efficiency controlled electroporation is performed using a device which includes one or more capillaries. The method of controlled electroporation comprises the steps of: 1) placing the one or more cells in an electroporation device comprising at least one elongate capillary having a lumen comprising a first end and a second end, where both the first end and the second end open into reservoirs and where the one or more cells can flow through the lumen of the at least one capillary and into the reservoirs; 2) contacting the one or more cells with a nucleic acid molecule comprising one or more DNA elements capable of opening chromatin and/or maintaining chromatin in an open state operably linked to a nucleotide sequence encoding the recombinant protein; 3) contacting the one or more cells with an electric current such that the current passes through the one or more cells; 4) monitoring the ratio between the current and voltage in the electroporation device; and 5) adjusting the magnitude of the local field strength to a field strength suitable to achieve electroporation of the one or more cells.

In some embodiments, the diameter of the lumen of a capillary is no greater than about 20% of the diameter of a cell in the lumen. In some embodiments, the diameter of the lumen of the capillary is no greater than about 20% of the diameter of a plurality of cells (e.g., the perimeter around a group of cells in the lumen).

In various controlled electroporation methods described herein, the optimal local field strength suitable for achieving the electroporation of a particular cell type can be readily determined using known methods in the art, e.g., by assaying for a change (e.g., an increase) in cell diameter over time when the cell is exposed to varying field strengths. In some embodiments, the local field strength which is used in the methods of the invention is about 150-500 V/cm. In other embodiments, the local field strength which is used in the methods of the invention is about 250-400 V/cm. In a particular embodiment, local field strength used in the methods of the invention is about 400 V/cm (e.g., in case of CHO cells).

In other embodiments of the invention transfection may be performed using a chemical reagent such as calcium phosphate as precipitant, or cationic lipids and the like, e.g. Lipofectamine™ (INVITROGEN, Carlsbad, Calif.).

Without wishing to be bound by theory, it is contemplated that any suitable method of transfection can be used in the methods of the invention, so long as it is capable of achieving at least 50% or more, or at least 60% or more, or at least 70% or more, or at least 80% or more, or at least 90% or more, or at least 95% or more, or at least 99% or more of the cells being transfected. Additional exemplary methods which may be used in the methods of the invention include, e.g., use of magnetic nanoparticles (e.g., see kits sold by OZ Biosciences) and nanoparticle transfection (e.g., see kits sold by SIGMA-ALDRICH). In a particular embodiment, any method capable of achieving transfection of at least 70% of the cells is used in the methods of the invention. In another embodiment, any method capable of achieving transfection of at least 80% of the cells is used in the methods of the invention.

### VI. CELL CULTURE MEDIA

Subsequent to electroporation, the cells are grown in a suitable medium to facilitate protein expression and secretion into the medium. Any suitable culture medium or feed medium suitable for cell growth and protein production may be used in the methods of invention. Suitable culture or feed mediums are chosen for their compatibility with the host cells and polypeptide of interest. Suitable culture or feed mediums are well known in the art and include, but are not limited to, commercial media such as Ham's F10 (SIGMA), Minimal Essential Medium (SIGMA), RPMI-1640 (SIGMA), and Dulbecco's Modified Eagle's Medium SIGMA) are suitable for culturing the animal cells.

In addition, any of the media described in Ham and Wallace, Meth. Enz., 58:44 (1979), Barnes and Sato, Anal. Biochem., 102:255 (1980), U.S. Pat. No. 4,767,704; 4,657,866; 4,927,762; or 4,560,655; WO 90/03430; WO 87/00195; U.S. Pat. No. Re. 30,985; or U.S. Pat. No. 5,122,469, the disclosures of all of which are incorporated herein by reference, may be used as culture or feed media for the host cells. Any of these media may be supplemented with additional components to meet the specific needs of the cells being culture.

### VII. CELL CULTURE METHODS

A recombinant protein of interest may be produced using any scheme or routine that may be suitable for a particular cell-type and the particular production plan desired. Therefore, it is contemplated that either a single-step or multiple-step culture procedure may be used in the methods of the invention. For example, in a single-step culture, the cells are inoculated into a culture environment and the subsequent addition of any nutrients or supplements is employed during a single production phase of the cell culture. Alternatively, a multi-stage culture may be used. In the multi-stage culture, cells may be cultivated in a number of steps or phases. For instance, cells may be grown in a first step or growth phase culture wherein cells, possibly removed from storage, are inoculated into a medium suitable for promoting growth and high viability. The cells may be maintained in the growth phase for a suitable period of time by the addition of fresh medium to the host cell culture. In a particular embodiment, the cells are grown in a multi-stage culture comprising one or more growth stages and a production stage. In some embodiments of the methods of the invention, the growth stage includes from about a 5 L culture volume to about a 200 L culture volume, while the production phase includes about a 15000 L culture volume.

Various cell culture conditions such as, for example, osmolality, temperature and pH may be controlled to obtain optimal protein production (e.g., a high titer) over the duration of the cell culture process and also to reduce batch variability. Such conditions may either be controlled at the growth phase or the production phase of the cell culture process or at both phases.

Additionally, it is contemplated that any suitable mode of culturing cells (e.g., fed-batch or continuous) may be used in the methods of the present invention. In some embodiments of the methods of the present invention, fed-batch or continuous cell culture conditions are used to enhance growth of the mammalian cells in the growth phase of the cell culture. In other embodiments of the methods of the present invention, a bulk cell culture method is devised for cell growth. During fed-batch, or continuous cell culture conditions, the growth phase cells are grown under conditions and for a period of time that is suitable for maximum growth. Culture conditions, such as temperature, pH, osmolality, dissolved oxygen (DO₂), and the like, that are optimal for a particular cell type would be apparent to one of ordinary skill in the art or can be readily determined by one of ordinary skill in the art.

### VIII. KITS

Also encompassed by the present invention are kits for producing a high titer of a recombinant protein. In some embodiments, a kit according to the invention comprises: a) a nucleic acid molecule comprising a DNA element capable of opening chromatin and/or maintaining chromatin in an open state operably linked to a multiple cloning site suitable for cloning a nucleotide sequence encoding the recombinant protein; and b) a device or reagent for performing high efficiency transfection, along with instructions for use.

In some embodiments, a kit according to the invention includes a device for performing controlled electroporation. In some embodiments, a kit according to the invention further comprises a means for monitoring the ratio between current and voltage. In some embodiments, the controlled electroporation is performed at a local field strength of about 400 V/cm.

In some embodiments, a kit according to the invention further comprises a cell line (e.g., comprising a plurality of cells) suitable for introducing the nucleic acid molecule. In some embodiments, the cell line comprises a plurality of mammalian cells (e.g., CHO cells).

In further embodiments, a kit according to the invention includes one or more nanoparticles suitable for transfection of nucleic acid molecules.

In some embodiments, kits featured herein include instructions and/or promotional materials including details regarding using a transfection device or reagent.

The specification is most thoroughly understood in light of the teachings of the references cited within the specification which are hereby incorporated by reference. The embodiments within the specification provide an illustration of embodiments in this invention and should not be construed to limit its scope. The skilled artisan readily recognizes that many other embodiments are encompassed by this invention. All publications and invention are incorporated by reference in their entirety. To the extent that the material incorporated by reference contradicts or is inconsistent with the present specification, the present specification will supercede any such material. The citation of any references herein is not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with a true scope and spirit of the invention being indicated by the following claims.

What is claimed is:

1. A method of producing high titer of a recombinant protein in cell culture without gene amplification, the method comprising: introducing into one or more cells, a nucleic acid molecule comprising one or more nucleotide sequences capable of opening chromatin and/or maintaining chromatin in an open state operably linked to a nucleotide sequence encoding the recombinant protein, wherein the nucleic acid molecule is introduced into the one or more cells using high efficiency transfection.

2. The method of claim 1, wherein the high efficiency transfection comprises controlled electroporation comprising the steps of: 1) placing the one or more cells in an electroporation device comprising a barrier having one or more openings suitable for receiving the one or more cells; 2) securing the one or more cells in the one or more openings; 3) contacting the one or more cells with the nucleic acid molecule; 4) contacting the one or more cells with an electric current such that the current passes through the cell; 5) monitoring the ratio between the current and voltage in the electroporation device; and 6) adjusting the magnitude of the local field strength to a value suitable to achieve electroporation of the one or more cells.

3. The method of claim 2, wherein the one or more cells are contacted with the nucleic acid molecule prior to being contacted with the electric current.

4. The method of claim 2, wherein the one or more cells are contacted with the electric current prior to being contacted with the nucleic acid molecule.

5. The method of claim 2, wherein the one or more cells are contacted with the nucleic acid molecule concurrently with the electric current.

6. The method of claim 1, wherein the high efficiency transfection comprises controlled electroporation comprising the steps of: 1) placing the one or more cells in an electroporation device comprising at least one elongate capillary having a lumen comprising a first end and a second end, wherein both the first end and the second end open into reservoirs and wherein the one or more cells can flow through the lumen of the at least one capillary and into the reservoirs; 2) contacting the one or more cells with a nucleic acid molecule comprising one or more DNA elements capable of opening chromatin and/or maintaining chromatin in an open state operably linked to a nucleotide sequence encoding the recombinant protein; 3) contacting the one or more cells with an electric current such that the current passes through the one or more cells; 4) monitoring the ratio between the current and voltage in the electroporation device; and 5) adjusting the magnitude of the local field strength to a field strength suitable to achieve electroporation of the one or more cells.

7. The method of claim 6, wherein the one or more cells are contacted with the nucleic acid molecule prior to being contacted with the electric current.

8. The method of claim 6, wherein the one or more cells are contacted with the electric current prior to being contacted with the nucleic acid molecule.

9. The method of claim 6, wherein the one or more cells are contacted with the nucleic acid molecule concurrently with the electric current.

10. The method of claim 1, wherein the high efficiency transfection comprises introduction of the nucleic acid molecule into at least 80% of the cells.

11. The method of claim 1, wherein the high efficiency transfection comprises introduction of the nucleic acid molecule into at least 85% of the cells.

12. The method of claim 1, wherein the high efficiency transfection comprises introduction of the nucleic acid molecule into at least 90% of the cells.

13. The method of claim 1, wherein the high efficiency transfection comprises introduction of the nucleic acid molecule into at least 95% of the cells.

14. The method of claim 1, wherein the one or more cells are mammalian cells.

15. The method of claim 1, wherein the one or more cells are selected from the group consisting of one or more BHK21 cells, one or more CHO cells, one or more CHO-K1 cells, one or more CHO-DUXX cells, one or more NSO cells or one or more Sp2/0 cells.

16. The method of claim 1, wherein the one or more cells are Chinese Hamster Ovary cells (CHO cells).

17. The method of claim 1, wherein the recombinant protein is a therapeutic protein.

18. The method of claim 1, wherein the recombinant protein is an antibody or an antigen-binding fragment thereof.

19. The method of claim 1, wherein the recombinant protein is a monoclonal antibody.

20. The method of claim 1, wherein the one or more DNA elements capable of opening the chromatin and/or maintaining the chromatin in an open state are chosen from: (a) one or more an extended methylation-free CpG islands; (b) one or more matrix attachment regions; (c) one or more stabilizing and antirepressor regions; and (d) any combinations of (a)-(c).

21. The method of claim 1, wherein the DNA element capable of opening chromatin and/or maintaining chromatin in an open state is naturally occurring.

22. The method of claim 1, wherein the DNA element capable of opening chromatin and/or maintaining chromatin in an open state is artificially synthesized.

23. The method of claim 1, wherein the DNA element capable of opening chromatin and/or maintaining chromatin in an open state is a combination of naturally occurring and artificially synthesized DNA elements.

24. The method of claim 20, wherein the one or more extended methylation-free CpG islands are derived from the promoter region of one or more ubiquitously expressed genes.

25. The method of claim 24, wherein the one or more ubiquitously expressed genes are chosen from human hnRNPA2, mouse hnRNPA2, human TBP, mouse TBP, human rpS3 and mouse rpS3.

26. The method of claim 1, wherein the nucleic acid molecule further comprises one or more of: (a) a nucleotide sequence capable of enhancing translation; (b) a nucleotide sequence capable of increasing secretion; and (c) a nucleotide sequence capable of increasing mRNA stability, operably linked to the nucleotide sequence encoding the recombinant protein.

27. The method of claim 2, wherein the barrier comprises a dielectric material.

28. The method of claim 2, wherein at least 75%, or at least 80%, or at least 85%, or at least 90%, or at least 95% of the openings are plugged by the one or more cells.

28. The method of claim 2, wherein the diameter of the one or more openings is smaller than the diameter of the one or more cells.

29. The method of claim 2, wherein the diameter of the one or more openings is substantially the same as the diameter of the one or more cells.

30. The method of claim 6, wherein the diameter of the one or more cells is at least 80% of the diameter of the lumen of the at least one capillary.

31. The method of claim 1, wherein the nucleic acid molecule is a vector.

32. The method of claim 30, wherein the vector is a plasmid.

33. The method of claim 30, wherein the vector is a viral vector.

34. The method of claim 2, wherein the cell is secured to the opening by the application of pressure.

35. The method of claim 2, wherein the cell is secured to the opening by the application of pressure.

36. The method of claim 2, wherein the electroporation device comprises two chambers, each suitable for receiving a buffer.

37. The method of claim 2, wherein each of the two chambers comprises the same buffer.

38. The method of claim 36, wherein each of the two chambers comprises a different buffer.

39. The method of claim 1, wherein the method does not include a selection step.

40. A kit for producing a high titer of a recombinant protein comprising: a) a nucleic acid molecule comprising one or more DNA elements capable of opening chromatin and/or maintaining chromatin in an open state operably linked to a multiple cloning site suitable for cloning a nucleotide sequence encoding the recombinant protein; and b) a device or reagent for performing high efficiency transfection, and instructions for use.

41. The kit of claim 41, wherein the device is a controlled electroporation device.

42. The kit of claim 41, further comprising a means for monitoring the ratio between current and voltage.

43. The kit of claim 40, further comprising a cell line suitable for introducing the nucleic acid molecule.

44. The kit of claim 43, wherein the cell line comprises a plurality of cells.

45. The method of claim 2 or 6, wherein the local field strength is about 250-400 V/cm.

46. The method of claim 1, wherein the high efficiency transfection comprises the use of nanoparticles.

47. The method of claim 46, wherein the high efficiency transfection comprises the use of magnetic nanoparticles.


SEQUENCE LISTING (14 sequences)

SEQ ID NO: 1 - DNA, 3048 bp, Mus musculus
accactaagccatctctccagccctgagtcatggttttagtgtgagaggcatcattgaattttctgagcacggccatcagggtagctggcacaggtcttcagatacaaggagatagttataagaaggcagccatggctgtggtgcactagaaatggagaaacagcttcatcaggtgacagaccagtctgactctgtcccatgattagaagccatcttgttacaaggtcaaaataagttcattcctgttttctgtaacacttgggtttgatcctgtcgtcaacccattttctggaatttgacatgttccatactccattataccctgacttccaccctgataagatgttctgccaagttcctgtgtagccaacattcccctggaaatctctcttcccttggaaaccacctagtcttagaaattttgagttatataaattccacttctatgtttgatgctattctttaaaactccactttagggagatagccctgtctgatagaaaataaaacttgcttaatttgtctaaaagattttaagtaatagtttttacttttgttccgtgggattagtacagggtgaaacagactcccgtgtttccagtgtgaagtgagccacacactgcagtacaagttatatcagcaggttctgcctctgcgcaatgaacttttgcttgtgtggacatcagggtctgtgtgaagggaaggtcctatggcctagttttatactattcaacagtctgtccccgaagccctggtgctttattattttgacaagcccctgctgctggtattccaccctgctgcgagtcaaaaaagttcctgtctcggaaaaacaaaacaaaacaaaacaaccaaaaaataaatttttttttcccacaggttctagtggaggtgctcactaccagaaatcctacaaataagcccatctcatggatcagggtttacctttgtaataatattaaatctgtgtgcatgtgcgcacgcatgtgttttatgcttgcatatatgtatacgcagccatggttttctactgtcccactcactctgtaacttactgagccatccagctggtcctctaaatacatttcaatgaaagttttcattagcgtgaacgtgaaggtggtaaaatctgttagtgtgtgcttatgcctgtggtttgcacctctagtctgaaggttgctcttttcaaattttttatttatttacgtttttacttctgagtcagaaactcataaaggccatggcctcgaattcgctatgtagtcaacgatgaccttaaacttgtgaccctctacttcgttagtgctggaaccccaagcttgctgagtacagagcactttcagaccggaactagatgtctacttcctgttccgcctacattacaggttgctaggttacaccccccctacgccgttttagacgcaaaacttcatttcccatgcaaaacttcatttcccatgaacacttgcaagggtcgccgcgctgcgcggcgtcattgctcccgccctatatacctacttccgcccgcgagccacttcctttcctttcagcggcgcgcggctgcaagatggcggtgcagatttccaagaagaggaaggtaagcgtctgggcccggttcgggagtccgccgcgggttctacaagtgccagggaggcctgtggctccgtaatcagtcctgtggagcgtctggggccgcctgccgtctcttcgagcctcggatggccgtagattgtgtattgggccggagccgggcgagtgctgtgtgcctgggcaagggagggacaaactcctcgagttctggaccgactcgaacaccgggcgcctccagttccggactagacacctttgagcgtttcttggtctccataatagtaatcctgtggcacagttagagggcgtgtgccatcagatctagtccagtctctttagtaagtgaagtttagcagtcccttctcttagtcgcgtgatcctgcaagtggccatagttgaaagcctacttactgactgctgccgtgttcactcgggacccggagctgcagcgtccctgtggttatcatttcatgggggaaaagtgtgcaggttgccaggtttagaaatagatggtctgtcgtttgtgcttatgcacacagatgataaacctgttttgagtcaggattcctctcctatccgaggtacaacttacagtcccagctgtacatgtgctacttggagacagatttttctttgtctcttgggtgtagattatgccgtagagcccttcgatgaagaggtgatgacgagtctgagtaggaagtgttgtctttgtccaagatgcctcactatgctgcgttctgtggcacagctgaaagcactgtggtcaaaagaaacttcctaaagatgaccaagaggcatttgtctgagaagggttgctgcttttctgtagggccattgggcttgctctgactaaccctgtcttcacctcagaggtaacttgtttcctttggttcagtttgtagctgatggcatcttcaaagctgagctgaatgaatttctcactcgggagctggctgaagatggctactctggagttgaagtccgagttacaccaaccaggacagaaatcattattttagccaccaggtagaaataccattgattgtcacctgtaaatattgtgtgtactgagatgctgtgtaaacttgggccaaccaagcagtaaatctggcctcagtgggtgtaactgctttgttagaactgcatttgggaagaacttaccttccatttaacctgtgtgctggcgttgtggtgggcggcaggtgggatcttgagtaaatggttgcgcttcccctctacaggacacagaatgttcttggggagaagggtcgtcggatcagagagttgaccgcagttgtccagaagcgctttggcttccctgaaggcagcgtagaggtgagttcctctgctttatctcccgggggttttagactgagttgggatgtggcttctgctatagaattgtacttctgaaaacctgacatggccagtgacagtcacaggtacttgatgct

SEQ ID NO: 2 - DNA, 1558 bp, Homo sapiens
ggccctccgcgcctacagctcaagccacatccgaagggggagggagccgggagctgcgcgcggggccgccggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccctgcggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgaccgtttctcacctgccttgcgccaaggcagggggcgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatcgatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcctcgg

SEQ ID NO: 3 - DNA, 4180 bp, Homo sapiens
ctaaaacagcttcacatggcttaaaataggggaccaatgtcttttccaatctaagtcccatttataataaagtccatgttccatttttaaaggacaatcctttcggtttaaaaccaggcacgattacccaaacaactcacaacggtaaagcactgtgaatcttctctgttctgcaatcccaacttggtttctgctcagaaaccctccctctttccaatcggtaattaaataacaaaaggaaaaaacttaagatgcttcaaccccgtttcgtgacactttgaaaaaagaatcacctcttgcaaacacccgctcccgacccccgccgctgaagcccggcgtccagaggcctaagcgcgggtgcccgcccccacccgggagcgcgggcctcgtggtcagcgcatccgcggggagaaacaaaggccgcggcacgggggctcaagggcactgcgccacaccgcacgcgcctacccccgcgcggccacgttaactggcggtcgccgcagcctcgggacagccggccgcgcgccgccaggctcgcggacgcgggaccacgcgccgccctccgggaggcccaagtctcgacccagccccgcgtggcgctgggggagggggcgcctccgccggaacgcgggtgggggaggggagggggaaatgcgctttgtctcgaaatggggcaaccgtcgccacagctccctaccccctcgagggcagagcagtccccccactaactaccgggctggccgcgcgccaggccagccgcgaggccaccgcccgaccctccactccttcccgcagctcccggcgcggggtccggcgagaaggggaggggaggggagcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatcgatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcctcggcgagcgtttcggcgggcagcaggtcctcgtgagcgaggctgcggagcttcccctccccctctctcccgggaaccgatttggcggccgccattttcatggctcgccttcctctcagcgttttccttataactcttttattttcttagtgtgctttctctatcaagaagtagaagtggttaactattttttttttcttctcgggctgttttcatatcgtttcgaggtggatttggagtgttttgtgagcttggatctttagagtcctgcgcacctcattaaaggcgctcagccttcccctcgatgaaatggcgccattgcgttcggaagccacaccgaagagcggggagggggggtgctccgggtttgcgggcccggtttcagagaagatatcaccacccagggcgtcgggccgggttcaatgcgagccgtaggacaaagaaaccattttatgtttttcctgtcttttttttcctttgagtaacggttttatctgggtctgcagtcagtaaaacgacagatgaaccgcggcaaaataaacataaattggaagccatcggccacgaggggcagggacgaaggtggttttctgggcgggggagggatattcgcgtcagaatcctttactgttcttaaggattccgtttaagttgtagagctgactcattttaagtaatgttgttactgagaagtttaacccttacgggacagatccatggacctttatagatgattacgaggaaagtgaaataacgattttgtccttagttatacttcgattaaaacatggcttcagaggctccttcctgtaatgcgtatggattgatgtgcaaaactgttttgggcctgggccgctctgtatttgaactttgttacttttctcattttgtttgcaatcttggttgaacattacattgataagcataaggtctcaagcgaagggggtctacctggttatttttctttgaccctaagcacgtttataaaataacattgtttaaaatcgatagtggacatcgggtaagtttggataaattgtgaggtaagtaatgagtttttgctttttgttagtgatttgtaaaacttgttataaatgtacattatccgtaatttcagtttagagataacctatgtgctgacgacaattaagaataaaaactagctgaaaaaatgaaaataactatcgtgacaagtaaccatttcaaaagactgctttgtgtctcataggagctagtttgatcatttcagttaattttttctttaatttttacgagtcatgaaaactacaggaaaaaaaatctgaactgggttttaccactactttttaggagttgggagcatgcgaatggagggagagctccgtagaactgggatgagagcagcaattaatgctgcttgctaggaacaaaaaataattgattgaaaattacgtgtgactttttagtttgcattatgcgtttgtagcagttggtcctggatatcactttctctcgtttgaggttttttaacctagttaacttttaagacaggtttccttaacattcataagtgcccagaatacagctgtgtagtacagcatataaagatttcagctctgaggtttttcctattgacttggaaaattgttttgtgcctgtcgcttgccacatggccaatcaagtagct

SEQ ID NO: 4 - DNA, 8285 bp, Homo sapiens
agcttcaatgtttttagcaccctctgtgtggaggaaaataatgcagattattctaattagtgtaatatctaaccacattaaaatatattacatagtaaactacactccataattttataaatttgactccccagggtaataaactagtctctagtctgctcaccttcaactgtacaataaagtcttggttcttttgaaatagacctcaaatgagacacctaaaattcaaagtgtctttacatttaaagacacctacaggaaagcaggtaaaagagccaggttaaaaacaaattctaaaaccacttagctgcagttaaacatatagtaaagatgcactaaagtttcttactctgtaaatcccttccacttcaggaaatattccactttcccattcactacacgtcgatctagtactttttccacgacaaattcttcaggctctgcctcttcaacttttttactctttccattctgtttttttcccattttttgctaaaataaaacaaaagagaaattaagaaatattcctcttgaattttgagcacattttcaaggctcaattgcttatattattatcacattcgacataaatttttacttctatatcccagggcagacaccttctggaaagattaaaagtcaacagacaataaaataaaagaatgctttatcttgttcatttagttcaaacttacaacccaccaccaaaataatacaataaaaaaacactatctggaaacagttatttttttccagtctttttttttgagacagggtctcacactcttgtcgcccaggctggagtgcagtggcgtgatctcagctcactgcaacctccgcctccccaggttcaagcagttctcatgcctcagcctccagagtagctgggattataggcggatgccaccatgccgggctaattttttttgtgtttttattagaaacagggtttcaccatgttgaccaggctggtctcaaactcctgacctgaagtgattcaccagcctgggcctcccaaagtgctggcattacaggcgtgagccactgcgcccggccctgtagtcttaaaagaccaagtttactaattttcactcattttaacaacactgcaacaaacaactatgcaggaagtacctaaagggtgatccagagaagcaagtagtagtgacaggtcttaggtgaacctatgacagaccttgtatccacccccagatggtaaaagccccagcccccttctcaattcaaatattaatgtcaaaagcatcaatgatacagagaaaagataaatgcagaatgaaaacatggttcaaaatcctgataccaactgcagggtcaactatagagaccactaggaggttcaattaaaggacaagattatttttccataatctctgtagataatatttcctaccacttagaacaaaactataaagctatcacttcaagagaccaacattacaaatttattttaattccctaaggtgaaaaaaatccttccttcctggtttctcaagagaaagtctatactggtaaccaaattcactttaaacaggcattttctttggtatgacactatttaagagaagcaggaaaccaacgtgaaccagctctttccaatggctcaagatttcctatgagaggactaaaaatggggaaaatttttatgagaggattaaaaatgggggaaaaaaaaccctgaaatggttaatcagaagatcctatgggctgagaaggaatccatcttaacatttcatcttaaagcaaatgctattgccgggggcagtggctcatgcctgtaatcccagcactttgggaggccgaggtgggcagatcatctgaggtcaggagtttgagaccagcctgaccaacatggagaaaccccgtttctactaaaaatacaaaattagccaggcatagtggtgcatgcctgtaatcccagctacttgggaggctgaggcaggagaactgcttgaacccaggaggcttaagttgcggtgagccaagatcacgccattgcactctagcctggacaacaagagaaaaactctgtctcaaaaaaacacaaaaacaaaaaacccaaatactatttaaaaaagataaaccttaattgctcaatcattaaagccatcccacaagtaaagcagcaagcagaaaaaagttaagaacacctcaaggctacagaaggacatttcaagctatgcaggcatatgaagtgtgcagacagatatgtaagaaaggcctcaagactgcaaaagggcatttcaagctatgcaagcatataggtaacacatacacacacacaaaataaaatcccctgaaatacaaaaacatgcagcaaacacctgacgtttttggataccatttctaagtcaggtgttatgattctcattagtcaagatacttgagtactgggcccaaacagctttctgccactgtacagtacaagaaggtaggaataatggtgggaggagcaaagacaaactgtaatagacagaagtgtatcagatacctatactacatgaaaaacaaaacagctactgccacaaagggagaaggctaacaaaataaagtcaacaataaatacagaaaatgaaaaggatacacactaaggtttacaaaaaaaaaaaggcagacaaaatgccatacagtattcattcactactatggcattcataagctagtttcaaatgctcactattttcttttatagtatatatttgccttaacccagcacttttttccaaaagtggatgagtcaaaataaatttcccattatttaagtgaaattaacagcacacatatctcacaacactaatgaatttttaaaatggaaagttaagaacttttaaagtggccaacctgtgatccttcacaaaataaactaaatacaataacagaccccaaaggctatcaattgcgtgcaaaaacaacttctgttttccagggtaaacagaatctaatgcagaatctaatgcagggtaaacagacttaatgcagaatctaatgatggcacaaattaaaaatcactaacgtgccctttttagtgtgaaacccagagagagcacatacaagccaaaaacaaatgctttattttacctaggagacattaacattcacctttacgtgtttaagattaatgcaatgttaaatattgtgaaaactgtaactttgaatttcatgatttttatgtgaatattccagggtttaaaaaaacttgtaacatgacatggctgaataagataaaaaaaaaatctagccttttctcccttctggctcatatttgcgatttcgatcattttgtttaaaaaacaaaacactgcaatgaattaaacttaatattcttctatgttttagagtaagttaaaacaagataaagtgaccaaagtaatttgaaagattcaatgacttttgctccaacctaggtgcacaaggtaccttgttctttaaattgggctttaatgaaaatacttctccagaattctggggatttaagaaaaattatgccaaccaacaagggctttaccattttatgtaacatttttcaacgctgcaaaaatgtgtgtatttctatttgaagataaaaatcctcagcaaaatccacattgcactgtccttcaaagattagccttctttgaactagttaagacactattaagccaagccagtatctccctgtaatgaattcgtttttctcttaattttcccctgtaatttacactgggagagctgggaaatatgtggatgtaaatttctcagccacagagatgcaaagttatactgtggggaaaaaaaacttgagttaaatccttacatattttaggttttcattaacttaccaatgtagttttgttggaggccattttttttattgcagacttgaagagctattactagaaaaatgcatgacagttaaggtaagtttgcatgacacaaaaaaggtaactaaatacaaattctgtttggattccaacccccaagtagagagcgcacactttcaaacgtgaatacaaatccagagtagatctgcgctcctacctacattgcttatgatgtacttaagtacgtgtcctaaccatgtgagtctagaaagactttactggggatcctggtacctaaaacagcttcacatggcttaaaataggggaccaatgtcttttccaatctaagtcccatttataataaagtccatgttccatttttaaaggacaatcctttcggtttaaaaccaggcacgattacccaaacaactcacaacggtaaagcactgtgaatcttctctgttctgcaatcccaacttggtttctgctcagaaaccctccctctttccaatcggtaattaaataacaaaaggaaaaaacttaagatgcttcaaccccgtttcgtgacactttgaaaaaagaatcacctcttgcaaacacccgctcccgacccccgccgctgaagcccggcgtccagaggcctaagcgcgggtgcccgcccccacccgggagcgcgggcctcgtggtcagcgcatccgcggggagaaacaaaggccgcggcacgggggctcaagggcactgcgccacaccgcacgcgcctacccccgcgcggccacgttaactggcggtcgccgcagcctcgggacagccggccgcgcgccgccaggctcgcggacgcgggaccacgcgccgccctccgggaggcccaagtctcgacccagccccgcgtggcgctgggggagggggcgcctccgccggaacgcgggtgggggaggggagggggaaatgcgctttgtctcgaaatggggcaaccgtcgccacagctccctaccccctcgagggcagagcagtccccccactaactaccgggctggccgcgcgccaggccagccgcgaggccaccgcccgaccctccactccttcccgcagctcccggcgcggggtccggcgagaaggggaggggaggggagcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcctcggcgagcgtttcggcgggcagcaggtcctcgtgagcgaggctgcggagcttcccctccccctctctcccgggaaccgatttggcggccgccattttcatggctcgccttcctctcagcgttttccttataactcttttattttcttagtgtgctttctctatcaagaagtagaagtggttaactattttttttttcttctcgggctgttttcatatcgtttcgaggtggatttggagtgttttgtgagcttggatctttagagtcctgcgcacctcattaaaggcgctcagccttcccctcgatgaaatggcgccattgcgttcggaagccacaccgaagagcggggagggggggtgctccgggtttgcgggcccggtttcagagaagatatcaccacccagggcgtcgggccgggttcaatgcgagccgtaggacaaagaaaccattttatgtttttcctgtcttttttttcctttgagtaacggttttatctgggtctgcagtcagtaaaacgacagatgaaccgcggcaaaataaacataaattggaagccatcggccacgaggggcagggacgaaggtggttttctgggcgggggagggatattcgcgtcagaatcctttactgttcttaaggattccgtttaagttgtagagctgactcattttaagtaatgttgttactgagaagtttaacccttacgggacagatccatggacctttatagatgattacgaggaaagtgaaataacgattttgtccttagttatacttcgattaaaacatggcttcagaggctccttcctgtaatgcgtatggattgatgtgcaaaactgttttgggcctgggccgctctgtatttgaactttgttacttttctcattttgtttgcaatcttggttgaacattacattgataagcataaggtctcaagcgaagggggtctacctggttatttttctttgaccctaagcacgtttataaaataacattgtttaaaatcgatagtggacatcgggtaagtttggataaattgtgaggtaagtaatgagtttttgctttttgttagtgatttgtaaaacttgttataaatgtacattatccgtaatttcagtttagagataacctatgtgctgacgacaattaagaataaaaactagctgaaaaaatgaaaataactatcgtgacaagtaaccatttcaaaagactgctttgtgtctcataggagctagtttgatcatttcagttaattttttctttaatttttacgagtcatgaaaactacaggaaaaaaaatctgaactgggttttaccactactttttaggagttgggagcatgcgaatggagggagagctccgtagaactgggatgagagcagcaattaatgctgcttgctaggaacaaaaaataattgattgaaaattacgtgtgactttttagtttgcattatgcgtttgtagcagttggtcctggatatcactttctctcgtttgaggttttttaacctagttaacttttaagacaggtttccttaacattcataagtgcccagaatacagctgtgtagtacagcatataaagatttcagctctgaggtttttcctattgacttggaaaattgttttgtgcctgtcgcttgccacatggccaatcaagta

SEQ ID NO: 5 - DNA, 1551 bp, Homo sapiens
gggcgtctccgccgcagctcggctcccgcgcgctcagcaccgccagcggcggccagatgcaggcggagcgaggagctcggggaggccgtgggcggcggccaggccgcggccggcctggcggagatcgccacagcgagcggcccggagccgcagcggcggtagccagaggcggcggcggaggcggcggcggggacggaggcggacgccggggccgtggccgtggccggggcttccgcggcgctcgcggaggccgaggaggaggaggcgccccgcgaggcagccgccgggagccgggaggctggggcgcaggggccagcgcgccggttgaagatgacagcgatgcagagacctatggagaagagaatgatgaacagggaaattattctaaaagaaagattgtctctaactgggatcgatatcaagatattgaaaaagaggtcaataatgaaagtggagagtcacagaggggaacagatttcagtgtcctccttagctctgcaggggactcattctcacagttccggtttgctgaggagaaagaatgggatagtgaagcttcttgtccaaaacagaattcagcattttatgtggatagtgagttattggttcgagcccttcaagagctgcctctctgcctccgactcaacgttgctgccgaactggtccagggtacagttcctttagaggttcctcaggtgaaaccaaagagaactgatgatggcaagggattagggatgcagttaaaggggcccttggggcctggaggaagggggcccatctttgagctgaaatctgtggctgctggctgccctgtgttgctgggcaaagacaacccaagcccgggtccttcaagggattctcagaaacccacttccccactgcagtcagcaggagaccatttggaagaagaactagatctgttgcttaatttagatgcacctataaaagagggagataacatcttaccagatcagacgtctcaggacctgaaatccaaggaagatggggaggtggtccaagaggaagaagtttgtgcaaaaccatctgtgactgaagaaaaaaacatggaacctgagcaaccaagtacctccaaaaatgttaccgaggaagagctggaagactggttggacagcatgatttcctaaaaaggggaaaaaaagtgcctgaagcaaatcttggttgccttctaacggcaggtgggcataaggctgtccttcaggaccagccagtttacaagcatgtctcaagctagtgtgttccattatgctcacagcagtaaatgcctacctctgtgtttgacatctgaaagaatacattgaagcagcttgttgcatttgtttttctggcttagtaatctaatagatttccttaagggcaggagatagactctggcccttgtttctagcctccttccttgcagtgtttacaacatagccagtgtttacagcatagcagatgctgctgctgattaagagaatagatgcaaacaaggcatgcatttggccaaaataaacaaatgctggtctgtccaaaaaaaaaaaaaaaaaaaaa

SEQ ID NO: 6 - protein, 362 aa, Homo sapiens
MQAERGARGGRGRRPGRGRPGGDRHSERPGAAAAVARGGGGGGGGDGGGRRGRGRGRGFRGARGGRGGGGAPRGSRREPGGWGAGASAPVEDDSDAETYGEENDEQGNYSKRKIVSNWDRYQDIEKEVNNESGESQRGTDFSVLLSSAGDSFSQFRFAEEKEWDSEASCPKQNSAFYVDSELLVRALQELPLCLRLNVAAELVQGTVPLEVPQVKPKRTDDGKGLGMQLKGPLGPGGRGPIFELKSVAAGCPVLLGKDNPSPGPSRDSQKPTSPLQSAGDHLEEELDLLLNLDAPIKEGDNILPDQTSQDLKSKEDGEVVQEEEVCAKPSVTEEKNMEPEQPSTSKNVTEEELEDWLDSMIS

SEQ ID NO: 7 - DNA, 1234 bp, Mus musculus
gtcgcgcgttctgctcgccggcggcctcatgcaggctgagcgcggggctaggggcggccgcgggcggcggggaggccgggagcggcccggaggggaccgagagccggtcggggcagcgacggcgctggcgagaggaggctgcggggacggaggcggccggcggggccgaggccggggcttccgccgaggccgaggaggtggcggcctgcgaggcggccgctgggagcctggaggccggggcggcggagccagcactcgggtggaagaagacagcgattcagagacctatggagaagagaatgatgagcaaggaaatttttctagaagaaagattgtctccaactgggatcgctatcaagatactgaaaaggaggtcaatggtgaaagtggagaatctcagcggggcacagacttcagtgtcctcctgagctctgcaggggactccttttcacagttccgatttgctgaggagaaagaatgggatggtgaaacttcatgtccaaaacagaattcagcactctacgtggacagtgagtcactggttcgagcccttgagcagctgcctcttgcagtcaggcttaatgttgcttcagaattgatccagaccacaattcctttagaacttccacaggtgaaaccaaggagaaacgatgatggcaaggagctgggcatgcatttaaggggacccatctctgagctcagatctgctgctggtgcttgccccaggtctctgggcagaggcagtctaaggcaaagccctttagaaggtttgcagaaagcacctaccccaacacagtcagtggcagaccacctggaagaagaactagatatgttgctgcatttagatgcacctgtgcaagaggaaggcattatctctccagaccagacatctcgggaccaggaaccagaaaaagatgggcaggtagcccaggaggaaacaggtcctgaaaaaccttctgtgaccagagagaagaatgtggaacctgagcagccaagcacatcgaagaatgtcaccgaggaagagctggaggactggctggacagcatgatttcctgaagcggggtcaagggggaagagtgcctgaagcaaatgctggttgccttctgtgtaatgagcccgtcttgtgaggacgggctcgtttaccagctaccccatgctaacacgtcctgttatgcttacagcagccaacacctacctctgtgtttgatggcatctgaatatatgtatgaggcc

SEQ ID NO: 8 - protein, 342 aa, Mus musculus
MQAERGARGGRGRRGGRERPGGDREPVGAATALARGGCGDGGGRRGRGRGFRRGRGGGGLRGGRWEPGGRGGGASTRVEEDSDSETYGEENDEQGNFSRRKIVSNWDRYQDTEKEVNGESGESQRGTDFSVLLSSAGDSFSQFRFAEEKEWDGETSCPKQNSALYVDSESLVRALEQLPLAVRLNVASELIQTTIPLELPQVKPRRNDDGKELGMHLRGPISELRSAAGACPRSLGRGSLRQSPLEGLQKAPTPTQSVADHLEEELDMLLHLDAPVQEEGIISPDQTSRDQEPEKDGQVAQEETGPEKPSVTREKNVEPEQPSTSKNVTEEELEDWLDSMIS

SEQ ID NO: 9 - DNA, 504 bp, Homo sapiens
atggagttgtggagtgagttacaaagttatcagaacctccgacgcttgctggagttggcttctgccagaacttccagctgttggagaatcctttttggctcaactttaactaatgtaatctatagagctaaggaggagtactcttcgcggtttgctgaccttttgtcgcataaccctggaatttttgcttctttgaatttggggcatcactcattttttcaagaaattgtgatcagaaatttagatttttcttctcctggccgtacggtttctgggcttgcttttatttgttttatattggatcaatggagcgcccaaactcatctgtcgcagggttatactctggattacatggcaatggctctgtggagaaccttgctacggaggaagagggtcttaggttgcttgccggcgcagcgtccgcacggtttggatccagtgcaggaagaggaggaggaggaggagaacctgagggccggcctggacccttcaacggaattgtaa

SEQ ID NO: 10 - protein, 167 aa, Homo sapiens
MELWSELQSYQNLRRLLELASARTSSCWRILFGSTLTNVIYRAKEEYSSRFADLLSHNPGIFASLNLGHHSFFQEIVIRNLDFSSPGRTVSGLAFICFILDQWSAQTHLSQGYTLDYMAMALWRTLLRRKRVLGCLPAQRPHGLDPVQEEEEEEENLRAGLDPSTEL

SEQ ID NO: 11 - DNA, 449 bp, Gallus gallus
aagcttctttggaaatacaccgacttgattgaagtctcttgaagatagtaaacagtacttacctttgatcccaatgaaatcgagcatttcagttgtaaaagaattccgcctattcataccatgtaatgtaattttacacccccagtgctgacactttggaatatattcaagtaatagactttggcctcaccctcttgtgtactgtattttgtaatagaaaatattttaaactgtgcatatgattattacattatgaaagagacattctgctgatcttcaaatgtaagaaaatgaggagtgcgtgtgcttttataaatacaagtgattgcaaattagtgcaggtgtccttaaaaaaaaaaaaaagtaatataaaaaggaccaggtgttttacaagtgaaatacattcctatttggaaaacagttacatttttatgaagattaccagcgct

SEQ ID NO: 12 - DNA, 660 bp, Gallus gallus
gcgctgctgactttctaaacataaggctgtattgtcttcctgtaccattgcatttcctcattcccaatttgcacaaggatgtctgggtaaactattcaagaaatggctttgaaatacagcatgggagcttgtctgagttggaatgcagagttgcactgcaaaatgtcaggaaatggatgtctctcagaatgcccaactccaaaggatttatatgtgtatatagtaagcagtttcctgattccagcaggccaaagagtctgctgaatgttgcgttgccggagacctgtatttctcaacaaggtaagatggtatcctagcaactgcggattttaatacattttcagcagaagtacttagttaatctctacctttagggatcgtttcatcatttttagatgttatacttgaaatactgcataacttttagctttcatgggttcctttttttcagcctttaggagactgttaagcaatttgctgtccaacttttgtgttggtcttaaactgcaatagtagtttaccttgtattgaagaaataaagaccatttttatattaaaaaatacttttgtctgtcttcattttgacttgtctgatatccttgcagtgctcattatgtcagttctgtcagatattcacacatcaaaacttaacgtgagctc

SEQ ID NO: 13 - DNA, 1668 bp, Gallus gallus
ggatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagctt

SEQ ID NO: 14 - DNA, 4672 bp, Artificial Sequence
  Description of Artificial Sequence Synthetic vector
aggtcactgtgacctagatccgcaggtcactgtgacctacatctgatatcatcgtcgacggtatcgataagcttcgaccgatccggccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatctcgagagatctcgaggcatgcaagcttggcattccggtactgttggtaaaatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtggccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaaaatgtaactgtattcagcgatgacgaaattcttagctattgtaatactgcgatgagtggcagggcggggcgtaatttttttaaggcagttattggtgcccttaaacgcctggtgctacgcctgaataagtgataataagcggatgaatggcagaaattcgccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgtcgagggggatccactagttctagagcggccgccaccgggatccataatataactgtaccaggttttggtttattacatgtgactgacggcttcctatgcgtgctcagaaaacggcagttgggcactgcactgcccggtgatggtgccacggtggctcctgccgccttctttgatattcactctgttgtatttcatctcttgttgccgatgaaaggatataacagtctctgaggaaatacttggtatttcttctgatcagcgtttttataagtaatgttgaatattggataaggctgtgtgtcctttgtcttgggagacaaagcccacagcaggtggtggttgggtggtggcagctcagtgacaggagaggtttttttgcctgttttttttgttgttttttttttttaagtaaggtgttcttttttcttagtaaaatttctactggactgtatgttttgacaggtcagaaacatttcttcaaaagaagaaccttttggaaactgtacagcccttttctttcattccctttttgctttctgtgccaatgcctttggttctgattgcattatggaaaacgttgatcggaacttgaggtttttatttatagtgtggcttgaaagcttggatagctgttgttacatgagataccttattaagtttaggccagcttgatgctttattttttttcctttgaagtagtgagcgttctctggtttttttcctttgaaactggcgaggcttagatttttctaatgggattttttacctgatgatctagttgcatacccaaatgcttgtaaatgttttcctagttaacatgttgataacttcggatttacatgttgtatatacttgtcatctgtgtttctagtaaaaatatatggcatttatagaaatacgtaattcctgatttccttttttttttatctctatgctctgtgtgtacaggtcaaacagacttcactcctatttttatttatagaattttatatgcagtctgtcgttggttcttgtgttgtaaggatacagccttaaatttcctagagcgatgctcagtaaggcgggttgtcacatgggttcaaatgtaaaacgggcacgtttgctgctgccttcccagatccaggacactaaactgcttctgcaactgaggtataaatcgcttcagatcccaggaagtgtagatccacgtgcatattcttaaagaagaatgaatactttctaaaatatgttggcataggaagcaagctgcatggatttatttgggacttaaattattttggtaacggagtgcataggttttaaacacagttgcagcatgctaacgagtcacagcatttatgcagaagtgatgcctgttgcagctgtttacggcactgccttgcagtgagcattgcagataggggtggggtgctttgtgtcgtgttgggacacgctgccacacagccacctcccgaacatatctcacctgctgggtacttttcaaaccatcttagcagtagtagatgagttactatgaaacagagaagttcctcagttggatattctcatgggatgtcttttttcccatgttgggcaaagtatgataaagcatctctatttgtaaattatgcacttgttagttcctgaatcctttctatagcaccacttattgcagcaggtgtaggctctggtgtggcctgtgtctgtgcttcaatcttttaagctt